(19) United States
(12) Patent Application Publication
(10) Pub. No.: US 2022/0017853 A1
(43) Pub. Date: Jan. 20, 2022

(54) COMPOSITIONS FOR STABILIZING BACTERIA AND USES THEREOF

(71) Applicant: Seres Therapeutics, Inc., Cambridge, MA (US)

(72) Inventors: Robert K. EVANS, Bangor, ME (US); Carl Michael PHILBROOK, Boston, MA (US); Brian Michael SCHUSTER, Arlington, MA (US); Lisa MARSHALL, Arlington, MA (US)

(73) Assignee: Seres Therapeutics, Inc., Cambridge, MA (US)

(21) Appl. No.: 17/311,262

(22) PCT Filed: Dec. 5, 2019
(86) PCT No.: PCT/US2019/064681

§ 371 Date: Jun. 4, 2021

Related U.S. Application Data
(60) Provisional application No. 62/775,697, filed on Dec. 5, 2018.

(51) Int. Cl. C12N 1/04; C12N 1/20; C12N 3/00; A61K 35/74

(52) U.S. Cl. CPC C12N 1/04; C12N 1/20; C12N 3/00; C12N 2500/60; C12N 2500/34; C12N 2500/32; C12N 2500/12; A61K 35/74

(57) ABSTRACT

Provided herein are compositions and formulations that are useful for stabilizing one or more bacteria (e.g., through drying). Methods of stabilizing the one or more bacteria are also disclosed.

### CROSS-REFERENCE TO RELATED APPLICATIONS

This PCT application claims the priority benefit of U.S. Provisional Application No. 62/775,697, filed Dec. 5, 2018, which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name: 4268_017PC01_Sequencelisting_ST25.txt; Size: 836,770 bytes; and Date of Creation: Dec. 5, 2019) filed with the application is herein incorporated by reference in its entirety.

### FIELD OF THE DISCLOSURE

The present disclosure relates to compositions and formulations that are useful for promoting the stability of dried bacteria.

### BACKGROUND OF THE DISCLOSURE

Lyophilization is a process used for preserving some biological molecules and can be used to prepare therapeutic compositions (e.g., peptides and proteins used as vaccines) that are to be reconstituted and administered to subjects. However, lyophilization of bacterial compositions has been challenging. The harsh conditions and stresses involved in the freeze drying process can negatively affect the structure, function, and viability of bacteria (Challener, C. A., BioPharm International 30(1): 32-35 (2017)). Furthermore, a lyophilized (or freeze-dried) formulation that works well for a specific species of bacterium, e.g., results in good stability, may not be effective for a different species, therefore making the process of producing mixtures of bacteria in which all species retain desired properties difficult.

Accordingly, there is a strong need for compositions and methods that can effectively and safely be used to dry bacteria for therapeutic use.

### SUMMARY OF THE DISCLOSURE

Provided herein is a composition comprising (i) one or more different OTUs of viable bacteria, (ii) urea, and (iii) one or more excipients selected from a cryoprotectant, an amino acid source, an antioxidant, a salt, a buffering agent, or combinations thereof. In some embodiments, the urea is present at a concentration (w/w) of between about 0.5% and about 1.0%.

In some embodiments, a composition disclosed herein comprises a cryoprotectant. In some embodiments, the cryoprotectant is a sugar. In certain embodiments, the sugar is a disaccharide. In some embodiments, the disaccharide is sucrose or trehalose. In certain embodiments, the disaccharide is sucrose and trehalose. In some embodiments, the sucrose and/or trehalose is present at a concentration of between about 5% and about 20%.

In some embodiments, a composition disclosed herein comprises an amino acid source. In some embodiments, the amino acid source is a collagen. In certain embodiments, the collagen is hydrolyzed collagen. In some embodiments, the amino acid source is a gelatin. In certain embodiments, the gelatin is a hydrolyzed gelatin. In further embodiments, the collagen is present at a concentration of about 3%. In some embodiments, the gelatin is present at a concentration between about 0.25% and about 4.0%. In some embodiments, the amino acid source is a casein or an albumin. In certain embodiments, the casein is hydrolyzed casein and/or the albumin is human serum albumin. In further embodiments, the casein and/or the albumin is present at a concentration of about 1%.

In some embodiments, a composition disclosed herein comprises an antioxidant. In certain embodiments, the antioxidant is cysteine. In some embodiments, the cysteine is present at a concentration of about 0.25%. In some embodiments, the antioxidant is ascorbic acid. In further embodiments, the ascorbic acid is present at a concentration of about 1.0%.

In some embodiments, a composition disclosed herein comprises a salt. In certain embodiments, the salt is a potassium salt. In further embodiments, the potassium salt is potassium chloride (KCl). In some embodiments, the KCl is present at a concentration of about 25 mM.

In some embodiments, a composition disclosed herein comprises a buffering agent. In some embodiments, the buffering agent is 4-(2-hydroxyethyl)-1-piperazineethanesulfonic acid (HEPES). In certain embodiments, the HEPES is present at a concentration between about 10 mM and about 100 mM.

In some embodiments, the viable bacteria present in a composition disclosed herein are anaerobes. In certain embodiments, the anaerobes have increased aerotolerance compared to corresponding anaerobes in a reference composition (e.g., lacks one of the excipients described herein, e.g., urea). In some embodiments, the anaerobes are facultative anaerobes. In further embodiments, the anaerobes are obligate anaerobes. In still further embodiments, the anaerobes are aerotolerant anaerobes. In some embodiments, the viable bacteria are aerobes.

In some embodiments, a composition disclosed herein comprises at least two OTUs of viable bacteria, wherein the at least two OTUs of viable bacteria comprises at least one facultative anaerobe, at least one obligate anaerobe, and/or at least one aerobe. In certain embodiments, the composition comprises at least one anaerobe (e.g., aerotolerant anaerobes) and at least one aerobe.

In some embodiments, the viable bacteria present in a composition of the present disclosure are spore-forming bacteria. In certain embodiments, the viable bacteria are in a spore form. In other embodiments, the viable bacteria are in a vegetative form. In some embodiments, the viable bacteria are in a mixture of spore-form and vegetative-form.

In some embodiments, the viable bacteria of a composition disclosed herein are from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In certain embodiments, the viable bacteria comprise a 16S rDNA sequence that is at least 97% identical to the 16S rDNA sequence set forth in SEQ ID NOs: 1-368.

Also disclosed herein is a dry powder comprising any of the compositions described in the present disclosure. In certain embodiments, the viable bacteria present in the dry powder are stable for at least 1 week, at least 2 weeks, at least 3 weeks, at least 1 month, at least 2 months, at least 3 months, at least 6 months, at least 1 year, or at least 2 years.

In some embodiments, a dry powder disclosed herein is encapsulated. In certain embodiments, the dry powder is reconstituted. In further embodiments, the dry powder is used to treat a gastrointestinal disorder.

Provided herein is also a therapeutic formulation comprising a dry powder disclosed herein. In some embodiments, the therapeutic formulation is administered orally, rectally, parenterally, topically, or mucosally. In certain embodiments, the therapeutic formulation is used to treat a subject with a microbiome-associated disease or disorder. In some embodiments, the microbiome-associated disease or disorder comprises an inflammatory bowel disease, bacterial infection (e.g., Clostridium difficile infection), obesity, diabetes, asthma/allergy, an autoimmune disease, a central nervous system (CNS) disease or disorder (e.g., Autism Spectral Disorder (ASD) and Parkinson's Disease), a cholestatic disease, gastric ulcers, chronic heart diseases, rheumatic diseases, kidney diseases, cancer, or any combination thereof.

### EMBODIMENTS

Embodiment 1. A formulation comprising urea and one or more excipients.

Embodiment 2. The formulation of Embodiment 1, wherein the one or more excipients comprise a cryoprotectant, an amino acid source, an antioxidant, a salt, a buffering agent, or combinations thereof.

Embodiment 3. The formulation of Embodiment 1 or 2, wherein the urea is present at a concentration (w/w) of between about 0.5% and about 1.0%.

Embodiment 4. The formulation of Embodiment 2, wherein the cryoprotectant is a sugar.

Embodiment 5. The formulation of Embodiment 4, wherein the sugar is a disaccharide.

Embodiment 6. The formulation of Embodiment 5, wherein the disaccharide is sucrose.

Embodiment 7. The formulation of Embodiment 5, wherein the disaccharide is trehalose.

Embodiment 8. The formulation of Embodiment 6, wherein the sucrose is present at a concentration of between about 5% and about 20%.

Embodiment 9. The formulation of any one of Embodiments 2 to 8, wherein the amino acid source is a collagen.

Embodiment 10. The formulation of Embodiment 9, wherein the collagen is hydrolyzed collagen.

Embodiment 11. The formulation of any one of Embodiments 2 to 8, wherein the amino acid source is a gelatin.

Embodiment 12. The formulation of Embodiment 11, wherein the gelatin is a hydrolyzed gelatin.

Embodiment 13. The formulation of Embodiment 9 or 10, wherein the collagen is present at a concentration of about 3%.

Embodiment 14. The formulation of Embodiment 11 or 12, wherein the gelatin is present at a concentration between about 0.25% and about 4.0%.

Embodiment 15. The formulation of any one of Embodiments 2 to 8, wherein the amino acid source is a casein.

Embodiment 16. The formulation of Embodiment 15, wherein the casein is hydrolyzed casein.

Embodiment 17. The formulation of Embodiment 15 or 16, wherein the casein is present at a concentration of about 1%.

Embodiment 18. The formulation of any one of Embodiments 2 to 17, wherein the antioxidant is cysteine.

Embodiment 19. The formulation of any one of Embodiments 2 to 17, wherein the antioxidant is ascorbic acid.

Embodiment 20. The formulation of Embodiment 18, wherein the cysteine is present at a concentration of about 0.25%.

Embodiment 21. The formulation of Embodiment 19, wherein the ascorbic acid is present at a concentration of about 1.0%.

Embodiment 22. The formulation of any one of Embodiments 2 to 21, wherein the salt is a potassium salt.

Embodiment 23. The formulation of Embodiment 22, wherein the potassium salt is potassium chloride (KCl).

Embodiment 24. The formulation of Embodiment 23, wherein the KCl is present at a concentration of about 25 mM.

Embodiment 25. The formulation of any one of Embodiments 2 to 24, wherein the buffering agent is 4-(2-hydroxyethyl)-1-piperazineethanesulfonic acid (HEPES).

Embodiment 26. The formulation of Embodiment 25, wherein the HEPES is present at a concentration between about 10 mM and about 100 mM.

Embodiment 27. A bacterial composition comprising (i) a formulation of any one of Embodiments 1 to 26 and (ii) one or more different OTUs of viable bacteria.

Embodiment 28. The bacterial composition of Embodiment 27, wherein the viable bacteria are anaerobes.

Embodiment 29. The bacterial composition of Embodiment 28, wherein the anaerobes have increased aerotolerance compared to corresponding anaerobes in a reference composition (e.g., lacks one of the excipients described herein, e.g., urea).

Embodiment 30. The bacterial composition of any one of Embodiments 27 to 29, wherein the viable bacteria are facultative anaerobes.

Embodiment 31. The bacterial composition of any one of Embodiments 27 to 29, wherein the viable bacteria are obligate anaerobes.

Embodiment 32. The bacterial composition of any one of Embodiments 27 to 29, wherein the viable bacteria are aerotolerant anaerobes.

Embodiment 33. The bacterial composition of Embodiment 27, wherein the viable bacteria are aerobes.

Embodiment 34. The bacterial composition of Embodiment 27, comprising at least two OTUs of viable bacteria, wherein the at least two OTUs of viable bacteria comprises at least one facultative anaerobe, at least one obligate anaerobe, and/or at least one aerobe.

Embodiment 35. The bacterial composition of Embodiment 34, comprising at least one anaerobe (e.g., aerotolerant anaerobes) and at least one aerobe.

Embodiment 36. The bacterial composition of any one of Embodiments 27 to 35, wherein the viable bacteria are spore-forming bacteria.

Embodiment 37. The bacterial composition of any one of Embodiments 27 to 36, wherein the viable bacteria are in a spore form.

Embodiment 38. The bacterial composition of any one of Embodiments 27 to 36, wherein the viable bacteria are in a vegetative form.

Embodiment 39. The bacterial composition of any one of Embodiments 27 to 36, wherein the viable bacteria are in a mixture of spore-form and vegetative-form.

Embodiment 40. A dry powder comprising urea and one or more excipients.

Embodiment 41. The dry powder of Embodiment 40, wherein the one or more excipients comprise a cryoprotectant, an amino acid source, an antioxidant, a salt, a buffering agent, or combinations thereof.

Embodiment 42. The dry powder of Embodiment 40 or 41, further comprising one or more different OTUs of viable bacteria.

Embodiment 43. The dry powder of Embodiment 42, wherein the viable bacteria are anaerobes.

Embodiment 44. The dry powder of Embodiment 43, wherein the anaerobes have increased aerotolerance compared to corresponding anaerobes in a reference dry powder (e.g., lacks one of the excipients described herein, e.g., urea).

Embodiment 45. The dry powder of any one of Embodiments 42 to 44, wherein the viable bacteria are facultative anaerobes.

Embodiment 46. The dry powder of any one of Embodiments 42 to 44, wherein the viable bacteria are obligate anaerobes.

Embodiment 47. The dry powder of any one of Embodiments 42 to 46, wherein the viable bacteria are aerotolerant anaerobes.

Embodiment 48. The bacterial composition of Embodiment 42, wherein the viable bacteria are aerobes

Embodiment 49. The dry powder of Embodiment 42 comprising at least two species of viable bacteria, wherein the at least two species of viable bacteria comprises at least one facultative anaerobe, at least one obligate anaerobe, and/or at least one aerobe.

Embodiment 50. The dry powder of Embodiment 49, comprising at least one anaerobe (e.g., aerotolerant anaerobes) and at least one aerobe.

Embodiment 51. The dry powder of any one of Embodiments 42 to 50, wherein the viable bacteria are spore-forming bacteria.

Embodiment 52. The dry powder of any one of Embodiments 42 to 51, wherein viable bacteria are in the spore-form.

Embodiment 53. The dry powder of any one of Embodiments 42 to 51, wherein the viable bacteria are in the vegetative form.

Embodiment 54. The dry powder of any one of Embodiments 42 to 51, wherein the viable bacteria are in a mixture of spore-form and vegetative-form.

Embodiment 55. The dry powder of any one of Embodiments 42 to 54, wherein the viable bacteria are stable for at least 1 week, at least 2 weeks, at least 3 weeks, at least 1 month, at least 2 months, at least 3 months, at least 6 months, at least 1 year, or at least 2 years.

Embodiment 56. The dry powder of any one of Embodiments 40 to 55, wherein the dry powder is encapsulated.

Embodiment 57. The dry powder of any one of Embodiments 40 to 56, wherein the dry powder is reconstituted.

Embodiment 58. The dry powder of any one of Embodiments 40 to 57, wherein the dry powder is used to treat a gastrointestinal disorder.

Embodiment 59. A therapeutic formulation comprising a dry powder of any one of Embodiments 40 to 58.

Embodiment 60. The therapeutic formulation of Embodiment 59, wherein the therapeutic formulation is administered orally, rectally, parenterally, topically, or mucosally.

Embodiment 61. The therapeutic formulation of Embodiment 59 or 60, wherein the therapeutic formulation is used to treat a subject with a microbiome-associated disease or disorder.

Embodiment 62. The therapeutic formulation of Embodiment 61, wherein the microbiome-associated disease or disorder comprises an inflammatory bowel disease, bacterial infection (e.g., Clostridium difficile infection), obesity, diabetes, asthma/allergy, an autoimmune disease, a central nervous system (CNS) disease or disorder (e.g., Autism Spectral Disorder (ASD) and Parkinson's Disease), a cholestatic disease, gastric ulcers, chronic heart diseases, rheumatic diseases, kidney diseases, cancer, or any combination thereof.

### BRIEF DESCRIPTION OF THE DRAWINGS/FIGURES

FIG. 1 shows a comparison of the short-term stability and dry yield of Bacteroides faecis after lyophilization using compositions containing (i) urea, (ii) no urea, or (iii) commercially-available microbial freeze drying composition. Short-term stability is represented by the thermal stability data after 1 week at 30° C. (white bars) and after 2 weeks at 30° C. (black bars). Dry yield is represented by the post-lyophilization stability data (striped bars). Both the thermal stability and post-lyophilization data are shown as log reduction in viability (CFU/mL). Composition #9 contained 0.5% (w/w) urea. Composition #12 was the commercially-available composition (OPS Freeze Drying Buffer). Compositions #1-8, 10, and 11 contained no urea. The excipients of the tested compositions are provided in Table 1 (see Example 1).

FIG. 2 shows the effect that Dextran 70 (Pharmacosmos) and Nutra® hydrolyzed gelatin (Nutra Food Ingredients) had on the short-term stability and dry yield of Bacteroides faecis after lyophilization. The thermal stability data show the short-term stability of lyophilized Bacteroides faecis after 1 week at 30° C. (white bars) and after 2 weeks at 30° C. (black bars). The post-lyophilization stability data (striped bars) represents the dry yield. Both the thermal stability and post-lyophilization data are shown as log reduction in viability (CFU/mL). Compositions #9-11 contained 2.5% (w/w) Dextran 70. Compositions #3-11 contained varying concentrations (1, 2, or 4%) of Nutra® hydrolyzed gelatin. Compositions #6-11 contained 0.5% (w/w) urea. Compositions #1, 2, and 12 contained no urea, Dextran 70, and Nutra® hydrolyzed gelatin. The excipients of the tested compositions are shown in Table 2 (see Example 2).

FIG. 3 shows a comparison of the effect of AppliChem® gelatin (PanReac AppliChem) or hydrolyzed casein (Hy-Case SF) on the short-term accelerated stability and dry yield of Bacteroides faecis after lyophilization. Short-term stability is represented by the thermal stability data after 1 week at 4° C. (white bars) and/or after 2 weeks at 4° C. (black bars). Dry yield is represented by the post-lyophilization stability data (striped bars). Both the thermal stability and post-lyophilization data are shown as log reduction in viability (CFU/mL). Composition #2 contained 1% (w/w) AppliChem® gelatin. Compositions #1 and 5 contained no AppliChem® gelatin and instead contained 1% Hy-Case SF. All the tested compositions contained 0.5% (w/w) urea. The left side of FIG. 3 (i.e., first three bars) provides data for Bacteroides faecis grown in the Peptone, Yeast, Glucose (PYG) Broth. The right side of FIG. 3 (i.e., the last two bars) provides data for Bacteroides faecis grown in ADM (Animal Derived Medium, i.e., growth medium) broth. The excipients of the tested compositions are provided in Table 3 (see Example 3).

FIG. 4 shows the effect of urea on the dry yield of Clostridium SP_D5 after lyophilization. The post-lyophilization data, which represents the dry yield, are shown as log reduction in viability (CFU/mL). Compositions #2-5 contained 0.5% (w/w) urea. Composition #1 contained no urea. Composition #6 was the commercially-available composition (OPS Freeze Drying Buffer). The components of the tested compositions are provided in Table 4 (see Example 4).

FIGS. 5A and 5B show the effect of urea on aerotolerance of oxygen-sensitive bacteria after lyophilization. FIG. 5A shows the data for lyophilized Eubacterium siraeum. FIG. 5B shows the data for lyophilized Roseburia hominis. In both FIGS. 5A and 5B, aerotolerance of the bacteria is shown as the maintenance of bacterial titer (CFU/mL) in the presence of oxygen over a period of approximately 3 hours. The aerotolerance of the lyophilized bacteria (circle) are compared to the aerotolerance of corresponding non-lyophilized bacteria (square). The horizontal dotted line represents the limit of detection of the assay.

FIGS. 6A and 6B show the long-term stability of bacteria from different families of Gram-positive bacteria when lyophilized with a freeze drying composition comprising 0.5% urea. FIG. 6A shows the dry yield of the different bacteria at various time points (i.e., 1, 2, 3, 4, or 6 months post-lyophilization), both at frozen temperatures (−65° C. and −20° C.) and refrigerated temperature (4° C.). The initial value provided for an individual bacteria strain corresponds to the dry yield that was measured at approximately two weeks post lyophilization. The time periods shown in parentheses along the x-axis refers to the time post lyophilization. FIG. 6B shows the moisture content of the different lyophilized bacteria compositions several months post lyophilization.

### DETAILED DESCRIPTION OF DISCLOSURE

### I. Formulations for Stabilizing Bacteria

Applicant has discovered that formulations comprising certain excipients can improve the stability of bacteria in a composition when dried. Surprisingly, Applicant has found that urea can increase the yield and/or improve the stability of the bacteria after drying. Accordingly, in some aspects, the present disclosure provides formulations that are useful for preparing bacterial compositions that have improved yield and/or stability when dried compared to formulations in the art. Applicant has further discovered that the formulations disclosed herein can increase the aerotolerance of oxygen sensitive bacterial species, such as Roseburia hominis and Eubacterium siraeum. Accordingly, the formulations provided herein are useful for multiple species and strains of bacteria, including anaerobes (e.g., obligate or aerotolerant anaerobes) and aerobes. As used herein, “formulation” refers to a combination of excipients that can be used to dry bacteria; a “composition” or “bacterial composition” refers to a formulation that includes bacteria. Formulations provided herein are useful for drying bacteria and/or storing bacteria.

In general, a formulation disclosed herein comprises urea. In some embodiments, the urea is at a concentration (w/w) of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 2.0%, about 3.0%, about 4.0%, or about 5.0% or more. In certain embodiments, the urea is at a concentration (w/w) of about 0.1%, about 0.2%, about 0.3%, about 0.4%, about 0.5%, about 0.6%, about 0.7%, about 0.8%, about 0.9%, or about 1.0%. In some embodiments, the urea is at a concentration (w/w) of about 0.5%. In other embodiments, the urea is at a concentration (w/w) of about 1.0%. In some embodiments, the urea is at a concentration (w/w) of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 2.0%, 3.0%, 4.0%, or 5.0% or more. In certain embodiments, the urea is at a concentration (w/w) of 0.1%, 0.2%, 0.3%, 0.4%, 0.5%, 0.6%, 0.7%, 0.8%, 0.9%, or 1.0%. In certain embodiments, the urea is at a concentration (w/w) of 0.5%. In other embodiments, the urea is at a concentration (w/w) of 1.0%.

In some embodiments, a formulation disclosed herein further comprises one or more additional excipients. In some embodiments, the one or more additional excipients comprise a cryoprotectant, an amino acid source, an antioxidant, a salt, a buffer, or any combinations thereof.

Accordingly, in some embodiments, a formulation provided herein comprises urea and a cryoprotectant. In certain embodiments, a formulation comprises urea, a cryoprotectant, and an amino acid source. In further embodiments, a formulation comprises urea, a cryoprotectant, and an antioxidant. In some embodiments, a formulation comprises urea, a cryoprotectant, and a salt. In other embodiments, a formulation comprises urea, a cryoprotectant, and a buffer. In certain embodiments, a formulation comprises urea, a cryoprotectant, an amino acid source, and an antioxidant. In some embodiments, a formulation comprises urea, a cryoprotectant, an amino acid source, and a salt. In further embodiments, a formulation comprises urea, a cryoprotectant, an amino acid source, and a buffer. In some embodiments, a formulation comprises urea, a cryoprotectant, an amino acid source, an antioxidant, and a salt. In certain embodiments, a formulation comprises urea, a cryoprotectant, an amino acid source, an antioxidant, and a buffer. In some embodiments, a formulation comprises urea, a cryoprotectant, an amino acid source, an antioxidant, a salt, and a buffer.

### Cryoprotectant

As used herein, the term “cryoprotectant” refers to a compound added to a biological sample to minimize or reduce the damage that can be caused by the drying process (e.g., freezing and/or thawing).

In some embodiments, a cryoprotectant is a sugar. As used herein, the term “sugar” refers to monosaccharides, disaccharides, and polysaccharides. In some embodiments, the sugar is a disaccharide, such as sucrose, trehalose, lactose, glucose, fructose, galactose, dextrose, maltose, cellobiose, chitobiose, or lactulose.

Applicants have discovered that, in certain embodiments, sucrose is a useful cryoprotectant that can be used with formulations disclosed herein. Accordingly, in some embodiments, a formulation disclosed herein comprises urea and sucrose.

In some embodiments, sucrose is present in a formulation disclosed herein at a concentration (w/w) of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% or more. In certain embodiments, the sucrose is at a concentration (w/w) between about 5% and about 20%. In other embodiments, the sucrose is at a concentration (w/w) of about 15%. In further embodiments, the sucrose is at a concentration (w/w) of about 12.5%. In certain embodiments, the sucrose is at a concentration of about 10%. In some embodiments, sucrose is present in a formulation disclosed herein at a concentration (w/w) of 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, or 25% or more. In certain embodiments, the sucrose is at a concentration (w/w) between 5% and 20%. In other embodiments, the sucrose is at a concentration (w/w) of 15%. In further embodiments, the sucrose is at a concentration (w/w) of 12.5%. In certain embodiments, the sucrose is at a concentration (w/w/) of 10%.

Applicants have also found that, in some embodiments, trehalose is an effective cryoprotectant that can be used with formulations disclosed herein. Therefore, in certain embodiments, a formulation disclosed herein comprises urea and trehalose.

In some embodiments, trehalose is present in the formulations disclosed herein at a concentration (w/w) of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% or more. In certain embodiments, trehalose is at a concentration (w/w) between about 5% and about 20%. In some embodiments, trehalose is present in a formulation disclosed herein at a concentration (w/w) of 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, or 25% or more. In certain embodiments, trehalose is at a concentration (w/w) between 5% and 20%. Trehalose can also be used effectively in combination with other sugars, e.g., sucrose. For instance, in some embodiments, a formulation of the present disclosure comprises urea, sucrose, and trehalose.

Additional non-limiting examples of cryoprotectant that can be used alone or in combination with a disaccharide, such as sucrose or trehalose, include dimethylsulfoxide (DMSO), hydroxyethyl starch, glycerol, polyethylene glycol, polyvinylpyrrolidone, methylcellulose, proline, a polymer, ectoin, and combinations thereof. Cryoprotectants are known in the art and described further, e.g., in Janz et al., Journal of Biomedicine and Biotechnology 2012; Mareschi et al. Experimental Hematology 2006 34:1563-1572; and Hunt et al. Transfus Med Hemother 2011 38:107-123, each of which is incorporated by reference herein in its entirety.

In some embodiments, a cryoprotectant disclosed herein (e.g., sucrose, trehalose) can serve as a bulking agent. Bulking agents can be added to a pharmaceutical product to add volume and mass to the product, thereby facilitating precise metering and handling thereof. Additional bulking agents that can be useful, including in combination with sucrose and/or trehalose, can be, but are not limited to, lactose, glucose, mannitol, sorbitol, raffinose, glycine, histidine, polyvinylpyrrolidone (PVP), dextran 40, albumin, and combinations thereof.

### Amino Acid Source

Amino acids can exhibit lyo- and cryoprotective effects similar to those of established stabilizers, such as sugars and/or polymers, but offer a greater diversity of chemical structures and physicochemical properties. Their ability to prevent protein aggregation is due to their multiple physicochemical properties including hydrophobic and ionic interactions, hydrogen bonding, side chain flexibility and molar volume effects. Accordingly, in some embodiments, a formulation disclosed herein comprises at least one amino acid source.

In some embodiments, an amino acid source is an albumin. Therefore, in certain embodiments, a formulation disclosed herein comprises urea, sucrose, and albumin. In further embodiments, a formulation comprises urea, trehalose, and albumin. In some embodiments, a formulation comprises, urea, sucrose, trehalose, and albumin.

In certain embodiments, the albumin is human albumin. In some embodiments, the human albumin is human serum albumin. In certain embodiments, albumin is present in the compositions of the present disclosure at a concentration (w/w) of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 3.0%, about 4.0%, or about 5.0% or more. In some embodiments, albumin is present at a concentration (w/w) of about 1.0%. In certain embodiments, albumin is present in the compositions of the present disclosure at a concentration (w/w) of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 3.0%, 4.0%, or 5.0% or more. In some embodiments, albumin is at a concentration (w/w) of 1.0%.

In some embodiments, an amino acid source is a gelatin. In certain embodiments, a formulation of the present disclosure comprises urea, sucrose, and gelatin. In other embodiments, a formulation comprises urea, trehalose, and gelatin. In further embodiments, a formulation comprises urea, sucrose, trehalose, and gelatin.

Non-limiting examples of gelatin, particularly hydrolyzed gelatin, that can be used as described herein include but are not limited to Nutra® hydrolyzed gelatin (Nutra Food Ingredients) and AppliChem® gelatin (PanReac AppliChem).

In some embodiments, gelatin (e.g., hydrolyzed gelatin) is present in a formulation disclosed herein at a concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 3.0%, about 4.0%, or about 5.0% or more. In certain embodiments, the gelatin (e.g., hydrolyzed gelatin) is at a concentration of about 0.25%. In other embodiments, the gelatin (e.g., hydrolyzed gelatin) is at a concentration of about 1.0%. In further embodiments, the gelatin (e.g., hydrolyzed gelatin) is at a concentration of about 2.0%. In still further embodiments, the gelatin (e.g., hydrolyzed gelatin) is at a concentration of about 4.0%.

In some embodiments, gelatin (e.g., hydrolyzed gelatin) is present in a formulation disclosed herein at a concentration of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 3.0%, 4.0%, or 5.0% or more. In certain embodiments, the gelatin (e.g., hydrolyzed gelatin) is at a concentration of 0.25%. In other embodiments, the gelatin (e.g., hydrolyzed gelatin) is at a concentration of 1.0%. In further embodiments, the gelatin (e.g., hydrolyzed gelatin) is at a concentration of 2.0%. In still further embodiments, the gelatin (e.g., hydrolyzed gelatin) is at a concentration of 4.0%.

In some embodiments, an amino acid source is a collagen (e.g., hydrolyzed collagen (e.g., VacciPro®)). Accordingly, in certain embodiments, a formulation comprises urea, sucrose, and collagen. In other embodiments, a formulation comprises urea, trehalose, and collagen. In some embodiments, a formulation comprises urea, sucrose, trehalose, and collagen.

In some embodiments, collagen (e.g., hydrolyzed collagen, e.g., VacciPro) is present in a composition disclosed herein at a concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 3.0%, about 4.0%, or about 5.0% or more. In certain embodiments, the collagen (e.g., hydrolyzed collagen) is at a concentration of about 3%.

In some embodiments, collagen (e.g., hydrolyzed collagen, e.g., VacciPro® (Gelita, Sergeant Bluff, IA) is present in a composition disclosed herein at a concentration of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 3.0%, 4.0%, or 5.0% or more. In certain embodiments, the collagen (e.g., hydrolyzed collagen) is at a concentration of 3%.

In some embodiments, an amino acid source is casein. Non-limiting examples of casein that can be used with the present formulations include hydrolyzed casein, e.g. Hy-Case SF (Kerry Corp.). In certain embodiments, a formulation provided herein comprises urea, sucrose, and casein. In other embodiments, a formulation comprises urea, trehalose, and casein. In some embodiments, a formulation comprises urea, sucrose, trehalose, and casein.

In some embodiments, a useful formulation as provided herein does not comprise albumin (e.g., human albumin), gelatin (e.g., hydrolyzed gelatin), collagen (e.g., hydrolyzed collagen), and/or casein (e.g. hydrolyzed casein).

### Antioxidant

Applicant has further discovered that an effective antioxidant that can be used with a formulation provided herein is cysteine. The term “antioxidant,” as used herein, refers to any substance that can inhibit oxidation. Therefore, in some embodiments, a formulation comprises urea, sucrose, and cysteine. In certain embodiments, a formulation comprises urea, trehalose, and cysteine. In further embodiments, a formulation comprises urea, sucrose, trehalose, and cysteine.

In certain embodiments, cysteine is present in a formulation disclosed herein at a concentration of about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1.0% or more. In some embodiments, cysteine is present at a concentration of about 0.25%. In certain embodiments, cysteine is present in a formulation disclosed herein at a concentration of 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, or 1.0% or more. In some embodiments, cysteine is present at a concentration of 0.25%.

In some embodiments, an effective antioxidant that can be used with a formulation of the present disclosure is ascorbic acid (vitamin C). In certain embodiments, a formulation of the present disclosure comprises urea, sucrose, and ascorbic acid. In other embodiments, a formulation comprises urea, trehalose, and ascorbic acid. In further embodiments, a formulation comprises urea, sucrose, trehalose, and ascorbic acid.

In some embodiments, ascorbic acid is at a concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0% or more. In certain embodiments, compositions disclosed herein comprise ascorbic acid at a concentration of about 1.0%. In some embodiments, ascorbic acid is at a concentration of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% or more. In certain embodiments, compositions disclosed herein comprise ascorbic acid at a concentration of 1.0%.

Non-limiting examples of other antioxidants that can be used in the present disclosure include: inulin, riboflavin, tocopherol (vitamin E), tocotrienol, carotenoids, carotene, provitamin A, vitamin A, propyl gallate, tertiary butylhydroquinone, butylated hydroxyanisole, butylated hydroxytoluene, sodium/potassium metabisulfite, catalase, superoxide dismutase, ubiquinol, glutathione, thiols, polyphenol, oxalic acid, phytic acid, tannins, eugenol, lipoic acid, uric acid, coenzyme Q, melatonin, and any combinations thereof.

### Salts

In some embodiments, a formulation disclosed herein for drying bacteria (e.g., anaerobic and aerobic) includes a salt. In certain embodiments, the salt is a potassium salt. For example, in certain embodiments, a formulation comprises urea, sucrose, and potassium salt. In other embodiments, a formulation comprises urea, trehalose, and potassium salt. In some embodiments, a formulation comprises urea, sucrose, trehalose, and potassium salt.

In some embodiments, the potassium salt is potassium chloride (KCl). In certain embodiments, the potassium chloride is present in a formulation disclosed herein at a concentration of about 5 mM, about 10 mM, about 15 mM, about 20 mM, about 25 mM, about 30 mM, about 35 mM, about 40 mM, about 45 mM, or about 50 mM. In some embodiments, the potassium chloride is present at a concentration of about 25 mM.

Non-limiting examples of other salts that can be included in the formulation disclosed herein include potassium iodide, sodium chloride, sodium sulfate, and combinations thereof. In some embodiments, a formulation disclosed herein can include more than one salt.

### Buffering Agent

Buffering agents useful for the present invention can be a weak acid or base used to maintain the pH of a solution near a chosen value after the addition of another acid or base. Suitable buffering agents can maximize the stability of a composition disclosed herein by maintaining pH control of the composition. Suitable buffering agents can also ensure physiological compatibility or optimize solubility. Rheology, viscosity, and other properties can also depend on the pH of the formulation or composition. Common buffering agents include, but are not limited to, histidine, citrate (e.g., sodium citrate), succinate, acetate (e.g., Tris acetate), phosphate (e.g., sodium phosphate), arginine HEPES, tartrate, Tris base, Tris-HCl, Tris-acetate, and combinations thereof.

In some embodiments, a buffering agent comprises L-histidine or mixtures of L-histidine with L-histidine hydrochloride with isotonicity agents and potentially pH adjustment with an acid or a base known in the art (e.g., HCl and/or NaOH). In certain embodiments, the buffering agent is L-histidine. Therefore, in some embodiments, a formulation disclosed herein comprises urea, sucrose, and L-histidine. In further embodiments, a formulation disclosed herein comprises urea, trehalose, and L-histidine. In still further embodiments, a formulation disclosed herein comprises urea, sucrose, trehalose, and L-histidine.

In further embodiments, the pH of a formulation or composition is maintained between about 6 and about 8, or between about 6.5 and about 7.5. In some embodiments, the pH is maintained between 6 and 8, or between 6.5 and 7.5. In certain embodiments, the pH of a formulation or composition disclosed herein is 6.5. In other embodiments, the pH of a formulation or composition disclosed herein is 6.0. In further embodiments, the pH of a formulation or composition disclosed herein is 7.0.

In some embodiments, a buffering agent comprises HEPES. For instance, in certain embodiments, a formulation comprises urea, sucrose, and HEPES. In some embodiments, a formulation comprises urea, trehalose, and HEPES. In further embodiments, a formulation comprises urea, sucrose, trehalose, and HEPES.

In certain embodiments, a formulation disclosed herein comprises HEPES at a concentration of about 1 mM, about 2 mM, about 3 mM, about 4 mM, about 5 mM, about 6 mM, about 7 mM, about 8 mM, about 9 mM, about 10 mM, about 15 mM, about 20 mM, about 30 mM, about 40 mM, about 50 mM, about 60 mM, about 70 mM, about 80 mM, about 90 mM, about 100 mM, about 150 mM, or about 200 mM. In certain embodiments, the HEPES is present at a concentration between about 10 mM and about 100 mM. In further embodiments, the HEPES is present at a concentration of about 50 mM. In some embodiments, a formulation disclosed herein comprises HEPES at a concentration of 1 mM, 2 mM, 3 mM, 4 mM, 5 mM, 6 mM, 7 mM, 8 mM, 9 mM, 10 mM, 15 mM, 20 mM, 30 mM, 40 mM, 50 mM, 60 mM, 70 mM, 80 mM, 90 mM, 100 mM, 150 mM, or 200 mM. In certain embodiments, the HEPES is present at a concentration between 10 mM and 100 mM. In further embodiments, the HEPES is present at a concentration of 50 mM. In further embodiments, the pH of a formulation or composition is maintained between about 6 and about 8, or between about 6.5 and about 7.5. In some embodiments, the pH is maintained between 6 and 8, or between 6.5 and 7.5. In certain embodiments, the pH of a formulation or composition disclosed herein is 6.5. In other embodiments, the pH of a formulation or composition disclosed herein is 6.0. In further embodiments, the pH of a formulation or composition disclosed herein is 7.0.

In some embodiments, a formulation of the present disclosure comprises urea, sucrose, human albumin, cysteine, and HEPES. In certain embodiments, a formulation comprises 0.5% urea, 15% sucrose, 1% human albumin, 0.25% cysteine, 50 mM HEPES, and pH 7.0. In other embodiments, a composition comprises 1.0% urea, 15% sucrose, 1% human albumin, 0.25% cysteine, 50 mM HEPES, and pH 7.0.

In some embodiments, a formulation disclosed herein comprises collagen (e.g., hydrolyzed collagen (such as VacciPro®)) and does not contain human albumin. Accordingly, in some embodiments, a formulation comprises urea, sucrose, collagen, cysteine, HEPES, and does not comprise human albumin. In certain embodiments, collagen is present in the formulation at a concentration of about 3%. In certain embodiments, collagen is present at a concentration of 3%.

In some embodiments, a composition of the present disclosure comprises KCl and does not contain cysteine. For instance, in certain embodiments, a formulation disclosed herein comprises urea, sucrose, human albumin, KCl, HEPES, and does not contain cysteine. In other embodiments, a formulation comprises urea, sucrose, collagen, KCl, HEPES, and does not contain cysteine. In certain embodiments, KCl is present at a concentration of about 10 mM, about 20 mM, about 30 mM, about 40 mM, about 50 mM or more. In some embodiments, KCl is present at a concentration of about 25 mM. In further embodiments, KCl is present at a concentration of at least 10 mM, 20 mM, 30 mM, 40 mM, 50 mM or more. In some embodiments, KCl is present at a concentration of 25 mM.

In some embodiments, a formulation disclosed herein comprises any number of additional components described above. In certain embodiments, a formulation disclosed herein comprises two components described above (e.g., urea and a cryoprotectant). In other embodiments, a formulation comprises three components described above. In some embodiments, a formulation comprises four components described above. In further embodiments, a formulation comprises five components described above. In certain embodiments, a formulation comprises six components described above. In still further embodiments, a formulation disclosed herein comprises seven components described above. In some embodiments, a formulation comprises eight components described above. In certain embodiments, a formulation comprises nine components described above. In other embodiments, a formulation comprises ten components described above. In some embodiments, a formulation disclosed herein can additionally include any other pharmaceutically acceptable components known in the art. See, e.g., Pramanick S., et al., Pharma Times 45(3): 65-77 (2013); Mehmood Y., and Farooq U., Open Science Journal of Pharmacy and Pharmacology 3(3): 19-27 (2015), both of which are hereby incorporated by reference in their entirety. In certain embodiments, a formulation disclosed herein further comprises a reducing agent (e.g., sodium metabisulfite), a chelating agent (e.g., citric acid), an acidic amino acid (e.g., sodium glutamate), a basic amino acid (e.g., arginine), a neutral surfactant (e.g., poloxamer), a polymer (e.g., nonionic triblock copolymer, polyvinylpyrrolidone), or combinations thereof.

In some embodiments, a formulation for lyophilizing a bacteria composition disclosed herein comprises urea, sucrose, gelatin hydrolysate, ascorbic acid, potassium chloride, HEPES, and NaOH. In certain embodiments, a lyophilization formulation comprises about 0.5% urea, about 10% sucrose, about 3% gelatin hydrolysate, about 1% ascorbic acid, about 25 mM potassium chloride, about 50 mM HEPES, and sufficient amount of NaOH to adjust the pH of the formulation to about 7.0.

The temperature at which the composition subjected to a drying process disclosed herein (e.g., lyophilization) loses structural integrity is the “collapse temperature” (Tc). In general, when using a drying process involving a freezing step, it is desirable to dry a composition below the collapse temperature to produce a good quality cake for storage. Increasing the collapse temperature can accelerate the drying process. In certain embodiments, a formulation disclosed herein comprises one or more collapse temperature modifiers, such as gelatin (e.g., hydrolyzed gelatin), collagen (e.g., hydrolyzed collagen), casein (e.g., hydrolyzed casein), ficoll, hydroxyethyl starch, or dextran (e.g., Dextran 70). In some embodiments, a formulation comprises one or more tonicity modifiers (e.g., dextrose, glycerol, sodium chloride, glycerin, and mannitol).

### II. Compositions

In some aspects, disclosed herein are compositions, e.g., bacterial compositions, comprising a population of bacteria belonging to one or more families, classes, genera, species, strains, and/or OTUs, and a lyophilization formulation disclosed herein. In some embodiments, the bacteria are viable and remain viable after lyophilization.

In some embodiments, a bacterial composition of the present disclosure comprises a single bacterium. In other embodiments, a bacterial composition comprises 2 or more types of bacteria. Accordingly, in certain embodiments, a bacterial composition disclosed herein comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 50, or greater than 50 types of bacteria, as defined by strain, species, or operational taxonomic unit (OTU). The bacteria can be present in approximately equal amounts from each family, genus, species, or OTU. In other embodiments, the bacteria are present in varying amounts in the composition.

In some embodiments, a bacterial composition disclosed herein comprises anaerobic bacteria. For example, in certain embodiments, a bacterial composition provided herein comprises (i) one or more anaerobic bacteria, (ii) urea, (iii) cryoprotectant, and (iv) an amino acid source. In some embodiments, a composition comprises (i) one or more anaerobic bacteria, (ii) urea, (iii) sucrose, and (iv) gelatin hydrolysate. In some embodiments, a composition comprises (i) one or more anaerobic bacteria, (ii) about 0.5% urea, (iii) about 10% sucrose, and (iv) about 3% gelatin hydrolysate.

In certain embodiments, one or more of the anaerobic bacteria present in a composition disclosed herein are obligate anaerobes. In some embodiments, a bacterial composition provided herein comprises (i) one or more obligate anaerobes, (ii) urea, (iii) cryoprotectant, and (iv) an amino acid source. In some embodiments, a composition comprises (i) one or more obligate anaerobes, (ii) urea, (iii) sucrose, and (iv) gelatin hydrolysate. In some embodiments, a composition comprises (i) one or more obligate anaerobes, (ii) about 0.5% urea, (iii) about 10% sucrose, and (iv) about 3% gelatin hydrolysate.

In other embodiments, one or more of the anaerobic bacteria are facultative anaerobes. In some embodiments, a bacterial composition provided herein comprises (i) one or more facultative anaerobes, (ii) urea, (iii) cryoprotectant, and (iv) an amino acid source. In some embodiments, a composition comprises (i) one or more facultative anaerobes, (ii) urea, (iii) sucrose, and (iv) gelatin hydrolysate. In some embodiments, a composition comprises (i) one or more facultative anaerobes, (ii) about 0.5% urea, (iii) about 10% sucrose, and (iv) about 3% gelatin hydrolysate.

In further embodiments, one or more of the anaerobic bacteria are aerotolerant anaerobes. In some embodiments, a bacterial composition provided herein comprises (i) one or more aerotolerant anaerobes, (ii) urea, (iii) cryoprotectant, and (iv) an amino acid source. In some embodiments, a composition comprises (i) one or more aerotolerant anaerobes, (ii) urea, (iii) sucrose, and (iv) gelatin hydrolysate. In some embodiments, a composition comprises (i) one or more aerotolerant anaerobes, (ii) about 0.5% urea, (iii) about 10% sucrose, and (iv) about 3% gelatin hydrolysate.

In some embodiments, a bacterial composition disclosed herein comprises aerobic bacteria. In some embodiments, a bacterial composition provided herein comprises (i) one or more aerobic bacteria, (ii) urea, (iii) cryoprotectant, and (iv) an amino acid source. In some embodiments, a composition comprises (i) one or more aerobic bacteria, (ii) urea, (iii) sucrose, and (iv) gelatin hydrolysate. In some embodiments, a composition comprises (i) one or more aerobic bacteria, (ii) about 0.5% urea, (iii) about 10% sucrose, and (iv) about 3% gelatin hydrolysate.

In certain embodiments, a bacterial composition comprises at least one anaerobe (e.g., aerotolerant anaerobes) and at least one aerobe. In some embodiments, a bacterial composition provided herein comprises (i) one or more anaerobes and one or more aerobes, (ii) urea, (iii) cryoprotectant, and (iv) an amino acid source. In some embodiments, a composition comprises (i) one or more anaerobes and one or more aerobes, (ii) urea, (iii) sucrose, and (iv) gelatin hydrolysate. In some embodiments, a composition comprises (i) one or more anaerobes and one or more aerobes, (ii) about 0.5% urea, (iii) about 10% sucrose, and (iv) about 3% gelatin hydrolysate.

In some embodiments, anaerobic bacteria, when present in a bacterial composition disclosed herein, have increased aerotolerance (e.g., remains stable for at least 3 hours post-lyophilization in the presence of oxygen) compared to corresponding anaerobic bacteria in a reference composition (e.g., lacks one of the excipients described herein, e.g., urea).

In some embodiments, a bacterial composition disclosed herein comprises one or more bacteria from a family, genus, species, or OTU useful in treating a subject with a microbiome-related disease or disorder. In certain embodiments, the subject can have a dysbiosis, e.g., of the GI tract, an infection, be at risk for infection (for example, infection associated with antibiotic treatment, radiation, chemotherapy), or have another disease or disorder affected by the microbiome (for example, an inflammatory bowel disease (e.g., ulcerative colitis, Crohn's disease), obesity, diabetes, asthma/allergy, an autoimmune disease, a central nervous system (CNS) disease or disorder (e.g., Autism Spectral Disorder (ASD) or Parkinson's Disease), a cholestatic disease, gastric ulcers, chronic heart diseases, rheumatic diseases, kidney diseases, or cancer, e.g., melanoma). In certain embodiments, a bacterial formulation disclosed herein comprises one or more bacteria that are present with high prevalence and/or high abundance in healthy individuals compared to an individual having a disease or risk factor.

In some embodiments, a bacterial composition of the present disclosure comprises one or more commensal bacteria derived from a human. In some embodiments, the one or more bacteria are Firmicutes. In some embodiments, the bacterial composition comprises bacteria from the class Clostridia. In some embodiments, the bacterial composition comprises bacteria from the order Clostridiales. In some embodiments, the bacterial composition comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In certain embodiments, a bacterial composition comprises at least one, two, three, four, five, six, seven, eight, nine, ten, eleven, twelve, thirteen, fourteen, or all of the listed families. For example, in some embodiments, a bacterial composition comprises bacteria from one of the families listed above. In other embodiments, a bacterial composition comprises bacteria from two of the families listed above. In further embodiments, a bacterial composition comprises bacteria from three of the families listed above. In some embodiments, a bacterial composition comprises bacteria from four of the families listed above. In certain embodiments, a bacterial composition comprises bacteria from five of the families listed above. In further embodiments, a bacterial composition comprises bacteria from six of the families listed above. In still further embodiments, a bacterial composition comprises bacteria from seven of the families listed above. In some embodiments, a bacterial composition comprises bacteria from eight of the families listed above. In some embodiments, a bacterial composition comprises bacteria from nine of the families listed above. In some embodiments, a bacterial composition comprises bacteria from ten of the families listed above. In some embodiments, a bacterial composition comprises bacteria from eleven of the families listed above. In some embodiments, a bacterial composition comprises bacteria from twelve of the families listed above. In some embodiments, a bacterial composition comprises bacteria from thirteen of the families listed above. In some embodiments, a bacterial composition comprises bacteria from fourteen of the families listed above. In some embodiments, a bacterial composition comprises bacteria from all fifteen of the families listed above.

In some embodiments, a bacterial composition comprises a population of bacteria that has been purified from a biological material (e.g., fecal material, such as feces or materials isolated from the various segments of the small and/or large intestine) obtained from a mammalian donor subject (e.g., a healthy human or a human responsive to a treatment, such as an immuno-oncology treatment). In some embodiments, the biological material (e.g., fecal material) is obtained from multiple donors (e.g., 2, 3, 4, 5, 6, 7, 8, 9, 10, 15, 20, 25, 30, 35, 40, 45, 50, 75, 100, 200, 300, 400, 500, 750, 1000, or from greater than 1000 donors), and the materials are pooled prior to purification or after purification of the desired bacteria. In other embodiments, the biological material (sample) can be obtained from a single donor subject at multiple times and two or more samples pooled, e.g., 2, 3, 4, 5, 6, 7, 8, 9, 10, 15, 20, 25, 30, 32, 35, 40, 45, 48, 50, 100 samples from a single donor. Methods of making such preparations include treatment of the feces with chloroform, acetone, ethanol, and the like, see, e.g., PCT/US2014/014745 and U.S. Pat. No. 9,011,834, which are incorporated herein by reference in their entirety.

In some embodiments, a population of bacteria derived from feces is depleted in residual habitat products. “Residual habitat products” refers to material derived from the habitat of a microbiota within or on a human or animal excluding the microbiota. An individual's microbiota is in, for example, feces in the gastrointestinal tract, on the skin itself, in saliva, mucus of the respiratory tract, or secretions of the genitourinary tract, all of which contain biological and other matter associated with the microbial community. “Substantially free of residual habitat products” means that the bacterial composition contains a reduced amount of the biological matter associated with the microbial environment on or in the human or animal subject and is 100% free, 99% free, 98% free, 97% free, 96% free, or 95% free of any contaminating biological matter associated with the microbial community or the contaminating matter is below a level of detection. Residual habitat products can include abiotic materials (including undigested food) or it can include unwanted microorganisms. Substantially free of residual habitat products can also mean that the bacterial composition contains no detectable cells from a human or animal and that only microbial cells are detectable. In some embodiments, substantially free of residual habitat products can mean that the bacterial composition contains no detectable viral (including bacterial viruses (i.e., phage)), fungal, mycoplasmal contaminants. In other embodiments, it means that fewer than 1×10⁻²%, 1×10⁻³%, 1×10⁻⁴%, 1×10⁵%, 1×10⁻⁶%, 1×10⁻⁷%, or 1×10⁸% of the viable cells in the bacterial composition are human or animal, as compared to microbial cells. There are multiple ways to accomplish reduced presence of residual habitat products, none of which are limiting. Thus, contamination can be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate reduction of residual habitat products include genetic analysis (e.g., PCR, DNA sequencing), serology and antigen analysis, enzymatic and metabolic analysis, and methods using instrumentation such as flow cytometry with reagents that distinguish desired constituents from contaminants.

In some embodiments, a bacterial composition comprises both spore-forming bacteria and non-spore-forming bacteria. In certain embodiments, the spore-forming bacteria are Gram-positive bacteria (e.g., Clostridium bolteae, Roseburia hominis, Eubacterium siraeum, or Clostridium sp_D7). In some embodiments, the non-spore-forming bacteria are Gram-negative bacteria (e.g., Bacteroides faecis or Bacteroides sp_4_1_36). In certain embodiments, a bacterial composition comprises only spore-forming bacteria. In some embodiments, the spore-forming bacteria are all in the form of spores. In other embodiments, some of the spore-forming bacteria are in the form of spores, while other spore-forming bacteria are in a vegetative form. Non-limiting examples of other bacterial strains that can be included in a bacterial composition of the present disclosure include those listed in Table 4, Table 5, FIG. 13, FIG. 17, FIG. 30, FIG. 31, or FIG. 32 of International Publication No. WO 2019/227085 A1, which is herein incorporated by reference in its entirety. Additional bacteria (including combinations of bacteria) that can be used with the present disclosure are provided in International Publication Nos. WO 2019/191390 A2; WO 2019/191694 A1; WO 2014/082050 A1, WO 2014/121298 A2; WO 2014/121304 A1; WO 2014/121301 A1; WO 2014/145958 A2; WO 2014/121302 A2; WO 2014/153194 A2; WO 2015/077794 A1; WO 2015/095241 A2; WO 2017/091783 A2; WO 2017/008026 A1; WO 2019/036510 A1; WO 2019/089643 A1; WO 2019/070913 A1; WO 2015/179437 A1; WO 2016/086161 A1; WO 2017/041039 A1; and WO 2017/091753 A1, each of which is herein incorporated by reference in its entirety.

In some embodiments, a composition disclosed herein comprises (i) a population of bacteria, (ii) urea, (iii) a cryoprotectant, and (iv) an amino acid source, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In some embodiments, a composition disclosed herein comprises (i) a population of bacteria, (ii) urea, (iii) sucrose, and (iv) gelatin hydrolysate, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In some embodiments, a composition disclosed herein comprises (i) a population of bacteria, (ii) about 0.5% urea, (iii) about 10% sucrose, and (iv) about 3% gelatin hydrolysate, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae.

In some embodiments, a composition disclosed herein comprises (i) a population of bacteria, (ii) urea, (iii) a cryoprotectant, (iv) an amino acid source, (v) an antioxidant, (vi) a salt, and (vii) a buffering agent, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In certain embodiments, a composition comprises (i) a population of bacteria, (ii) urea, (iii) sucrose, (iv) gelatin hydrolysate, (v) ascorbic acid, (vi) potassium chloride, and (vii) HEPES, wherein the population of bacteria comprise bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In further embodiments, a composition comprises (i) a population of bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae.

In some embodiments, a bacterial composition comprises a 16S rDNA sequence that is at least 97% identical to the 16S rDNA sequence set forth in SEQ ID NOs: 1 to 398. Accordingly, in certain embodiments, a composition for lyophilization disclosed herein comprises (i) one or more bacteria comprising a 16S rDNA sequence that is at least 97% identical to the 16S rDNA sequence set forth in SEQ ID NOs: 1 to 398, (ii) urea, (iii) a cryoprotectant, and (iv) an amino acid source. In some embodiments, a composition comprises (i) one or more bacteria comprising a 16S rDNA sequence that is at least 97% identical to the 16S rDNA sequence set forth in SEQ ID NOs: 1 to 398, (ii) urea, (iii) sucrose, and (iv) gelatin hydrolysate. In further embodiments, a composition comprises (i) one or more bacteria comprising a 16S rDNA sequence that is at least 97% identical to the 16S rDNA sequence set forth in SEQ ID NOs: 1 to 398, (ii) about 0.5% urea, (iii) about 10% sucrose, and (iv) about 3% gelatin hydrolysate.

In some embodiments, a composition disclosed herein comprises (i) one or more bacteria comprising a 16S rDNA sequence that is at least 97% identical to the 16S rDNA sequence set forth in SEQ ID NOs: 1 to 398, (ii) urea, (iii) a cryoprotectant, (iv) an amino acid source, (v) an antioxidant, (vi) a salt, and (vii) a buffering agent. In some embodiments, a composition comprises (i) one or more bacteria comprising a 16S rDNA sequence that is at least 97% identical to the 16S rDNA sequence set forth in SEQ ID NOs: 1 to 398, (ii) urea, (iii) sucrose, (iv) gelatin hydrolysate, (v) ascorbic acid, (vi) potassium chloride, and (vii) HEPES. In further embodiments, a composition for lyophilization comprises (i) one or more bacteria comprising a 16S sequence that is at least 97% identical to the 16S rDNA sequence set forth in SEQ ID NOs: 1 to 398, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES. In some embodiments, a sufficient amount of NaOH is used to adjust the pH of the formulation to about 7.0.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NO: 115. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NO: 115. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NO: 115.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NO: 115. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NO: 115. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NO: 115.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NOs: 227 or 136. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NOs: 227 or 136. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NOs: 227 or 136.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NOs: 227 or 136. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NOs: 227 or 136. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NOs: 227 or 136.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NO: 188. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NO: 188. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NO: 188.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NO: 188. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NO: 188. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NO: 188.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NO: 116. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NO: 116. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NO: 116.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NO: 116. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NO: 116. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NO: 116.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NOs: 120-131, 103, 118, or 189. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NOs: 120-131, 103, 118, or 189. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NOs: 120-131, 103, 118, or 189.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NOs: 120-131, 103, 118, or 189. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NOs: 120-131, 103, 118, or 189. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NOs: 120-131, 103, 118, or 189.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NO: 112. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NO: 112. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NO: 112.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NO: 112. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NO: 112. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NO: 112.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NO: 113. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NO: 113. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NO: 113.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NO: 113. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NO: 113. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NO: 113.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NOs: 148-150, 105, 217, 214-216, 178, 184, 223, 199, 181, or 114. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NOs: 148-150, 105, 217, 214-216, 178, 184, 223, 199, 181, or 114. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NOs: 148-150, 105, 217, 214-216, 178, 184, 223, 199, 181, or 114.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NOs: 148-150, 105, 217, 214-216, 178, 184, 223, 199, 181, or 114. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NOs: 148-150, 105, 217, 214-216, 178, 184, 223, 199, 181, or 114. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NOs: 148-150, 105, 217, 214-216, 178, 184, 223, 199, 181, or 114.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NOs: 196, 107-111, 219, 153, 160, 161, 154-158, 132-135, 314-317, 205-209, 222, 104, 224, 106, 179, 180, 225, or 187. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NOs: 196, 107-111, 219, 153, 160, 161, 154-158, 132-135, 314-317, 205-209, 222, 104, 224, 106, 179, 180, 225, or 187. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NOs: 196, 107-111, 219, 153, 160, 161, 154-158, 132-135, 314-317, 205-209, 222, 104, 224, 106, 179, 180, 225, or 187.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NOs: 196, 107-111, 219, 153, 160, 161, 154-158, 132-135, 314-317, 205-209, 222, 104, 224, 106, 179, 180, 225, or 187. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NOs: 196, 107-111, 219, 153, 160, 161, 154-158, 132-135, 314-317, 205-209, 222, 104, 224, 106, 179, 180, 225, or 187. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NOs: 196, 107-111, 219, 153, 160, 161, 154-158, 132-135, 314-317, 205-209, 222, 104, 224, 106, 179, 180, 225, or 187.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NOs: 137-146, 190-194, 218, 200-204, 183, 166-177, 221, 197, 263, 159, 147, 152, 185, 226, or 212. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NOs: 137-146, 190-194, 218, 200-204, 183, 166-177, 221, 197, 263, 159, 147, 152, 185, 226, or 212. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NOs: 137-146, 190-194, 218, 200-204, 183, 166-177, 221, 197, 263, 159, 147, 152, 185, 226, or 212.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NOs: 137-146, 190-194, 218, 200-204, 183, 166-177, 221, 197, 263, 159, 147, 152, 185, 226, or 212. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NOs: 137-146, 190-194, 218, 200-204, 183, 166-177, 221, 197, 263, 159, 147, 152, 185, 226, or 212. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NOs: 137-146, 190-194, 218, 200-204, 183, 166-177, 221, 197, 263, 159, 147, 152, 185, 226, or 212.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NOs: 186 or 211. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NOs: 186 or 211. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NOs: 186 or 211.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NOs: 186 or 211. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NOs: 186 or 211. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NOs: 186 or 211.

In some embodiments, a composition disclosed herein comprises (i) one or more bacteria, (ii) urea, (iii) a cryoprotectant, and (iv) an amino acid source, wherein the one or more bacteria comprise a 16S rDNA sequence that is at least 97% identical to the 16S rDNA sequence set forth in SEQ ID NOs: 151, 182, 213, or 198. In certain embodiments, a composition comprises (i) one or more bacteria, (ii) urea, (iii) sucrose, and (iv) gelatin hydrolysate, wherein the one or more bacteria comprise a 16S rDNA sequence that is at least 97% identical to the 16S rDNA sequence set forth in SEQ ID NOs: 151, 182, 213, or 198. In further embodiments, a composition comprises (i) one or more bacteria, (ii) about 0.5% urea, (iii) about 10% sucrose, and (iv) about 3% gelatin hydrolysate, wherein the one or more bacteria comprise a 16S rDNA sequence that is at least 97% identical to the 16S rDNA sequence set forth in SEQ ID NOs: 151, 182, 213, or 198.

In some embodiments, a composition disclosed herein comprises (i) one or more bacteria, (ii) urea, (iii) a cryoprotectant, (iv) an amino acid source, (v) an antioxidant, (vi) a salt, and (vii) a buffering agent, wherein the one or more bacteria comprise a 16S rDNA sequence that is at least 97% identical to the 16S rDNA sequence set forth in SEQ ID NOs: 151, 182, 213, or 198. In certain embodiments, a composition comprises (i) one or more bacteria, (ii) urea, (iii) sucrose, (iv) gelatin hydrolysate, (v) ascorbic acid, (vi) potassium chloride, and (vii) HEPES, wherein the one or more bacteria comprise a 16S rDNA sequence that is at least 97% identical to the 16S rDNA sequence set forth in SEQ ID NOs: 151, 182, 213, or 198. In further embodiments, a composition comprises (i) one or more bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the one or more bacteria comprise a 16S rDNA sequence that is at least 97% identical to the 16S rDNA sequence set forth in SEQ ID NOs: 151, 182, 213, or 198.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 151, 196, 190, 191, 192, 193, 194, 120, 121, 122, 123, 124, 125, 126, 127, 128, 129, 130, 131, 136, 200, 201, 202, 203, 204, 148, 149, 150, 107, 108, 109, 110, 111, 105, 182, 219, 153, 115, 213, 166, 167, 168, 169, 170, 171, 172, 173, 174, 175, 176, 177, 214, 215, 216, 103, 178, 161, 154, 155, 156, 157, 158, 119, 132, 133, 134, 135, 314, 315, 316, 317, 117, 205, 206, 207, 208, 209, 220, 221, 222, 197, 263, 102, 118, 159, 198, 112, 184, 104, 223, 189, 186, 224, 106, 199, 147, 211, 179, 180, 152, 195, 185, 116, 225, 226, 210, 212, 181, 114, 187, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 151, 196, 190, 191, 192, 193, 194, 120, 121, 122, 123, 124, 125, 126, 127, 128, 129, 130, 131, 136, 200, 201, 202, 203, 204, 148, 149, 150, 107, 108, 109, 110, 111, 105, 182, 219, 153, 115, 213, 166, 167, 168, 169, 170, 171, 172, 173, 174, 175, 176, 177, 214, 215, 216, 103, 178, 161, 154, 155, 156, 157, 158, 119, 132, 133, 134, 135, 314, 315, 316, 317, 117, 205, 206, 207, 208, 209, 220, 221, 222, 197, 263, 102, 118, 159, 198, 112, 184, 104, 223, 189, 186, 224, 106, 199, 147, 211, 179, 180, 152, 195, 185, 116, 225, 226, 210, 212, 181, 114, 187, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 190, 191, 192, 193, 194, 200, 201, 202, 203, 204, 214, 215, 216, 178, 197, 263, 102, 104, 179, 180, 152, 210, 181, 196, 186, 106, 211, 212, 116, 187, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 190, 191, 192, 193, 194, 200, 201, 202, 203, 204, 214, 215, 216, 178, 197, 263, 102, 104, 179, 180, 152, 210, 181, 196, 186, 106, 211, 212, 116, 187, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 197, 263, 179, 180, 152, 116, 181, 187, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 197, 263, 179, 180, 152, 116, 181, 187, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 197, 263, 179, 180, 152, 116, 181, 187, 196, 200, 201, 202, 203, 204, 148, 149, 150, 103, 132, 133, 134, 135, 314, 315, 316, 317, 102, 118, 186, 106, 211, 195, 226, 210, 212, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 197, 263, 179, 180, 152, 116, 181, 187, 196, 200, 201, 202, 203, 204, 148, 149, 150, 103, 132, 133, 134, 135, 314, 315, 316, 317, 102, 118, 186, 106, 211, 195, 226, 210, 212, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 226, 212, 152, 186, 210, 195, 211, 102, 179, 180, 116, 118, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 226, 212, 152, 186, 210, 195, 211, 102, 179, 180, 116, 118, 106, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 212, 152, 186, 210, 195, 211, 103, 102, 179, 180, 147, 116, 106, 225, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 212, 152, 186, 210, 195, 211, 103, 102, 179, 180, 147, 116, 106, 225, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 212, 152, 186, 210, 223, 195, 211, 103, 102, 179, 180, 116, 106, 225, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 212, 152, 186, 210, 223, 195, 211, 103, 102, 179, 180, 116, 106, 225, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 159, 152, 186, 210, 223, 195, 211, 103, 102, 224, 179, 180, 116, 106, 225, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 159, 152, 186, 210, 223, 195, 211, 103, 102, 224, 179, 180, 116, 106, 225, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 159, 152, 186, 210, 195, 211, 103, 102, 224, 179, 180, 147, 116, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 159, 152, 186, 210, 195, 211, 103, 102, 224, 179, 180, 147, 116, 106, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 226, 152, 210, 195, 211, 103, 102, 179, 180, 147, 116, 106, 225, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 226, 152, 210, 195, 211, 103, 102, 179, 180, 147, 116, 106, 225, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 226, 212, 152, 186, 210, 195, 211, 103, 102, 224, 179, 180, 116, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 226, 212, 152, 186, 210, 195, 211, 103, 102, 224, 179, 180, 116, 106, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 226, 152, 186, 210, 195, 211, 102, 179, 180, 147, 116, 106, 225, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 226, 152, 186, 210, 195, 211, 102, 179, 180, 147, 116, 106, 225, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 152, 210, 195, 211, 103, 224, 179, 180, 116, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 152, 210, 195, 211, 103, 224, 179, 180, 116, 106, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 152, 210, 195, 211, 102, 179, 180, 147, 116, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 152, 210, 195, 211, 102, 179, 180, 147, 116, 106, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in 16S rDNA sequence set forth in SEQ ID NOs: 178, 187, 196, 197, 263, 226, 152, 210, 195, 103, 102, 179, 180, 147, 116, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 226, 152, 210, 195, 103, 102, 179, 180, 147, 116, 106, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 152, 210, 223, 195, 211, 102, 179, 180, 147, 116, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 152, 210, 223, 195, 211, 102, 179, 180, 147, 116, 106, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 152, 186, 210, 195, 103, 102, 224, 179, 180, 116, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 152, 186, 210, 195, 103, 102, 224, 179, 180, 116, 106, 181, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 212, 152, 186, 195, 211, 103, 102, 116, 106, 225, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 197, 263, 200, 201, 202, 203, 204, 212, 152, 186, 195, 211, 103, 102, 116, 106, 225, or combinations thereof.

In some embodiments, a composition for lyophilization disclosed herein comprises one or more bacteria, urea, sucrose, and gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 152, 186, 210, 195, 211, 103, 102, 224, 116, 106, 181, or combinations thereof. In certain embodiments, a composition for lyophilization comprises one or more bacteria, about 0.5% urea, about 10% sucrose, and about 3% gelatin hydrolysate, wherein the one or more bacteria comprise a 16S rDNA sequence that is at least 97% identical to the 16S rDNA sequence set forth in SEQ ID NOs: 178, 187, 196, 200, 201, 202, 203, 204, 152, 186, 210, 195, 211, 103, 102, 224, 116, 106, 181, or combinations thereof.

When drying bacteria (e.g., as described herein), Applicant has discovered certain advantages, in some cases, to using bacteria in early stationary phase when producing lyophilized bacterial compositions. For instance, in certain embodiments, using bacteria in early stationary phase allows for greater stability when lyophilized using a formulation disclosed herein, compared to bacteria from a different stage of growth phase, e.g., lag phase or log phase. As used herein, the term “early stationary phase” refers to the stage of bacterial growth that immediately follows the log phase (sometimes called the logarithmic or exponential phase, which is characterized by cell doubling). The early stationary phase can be defined as a state of little to no net growth (i.e., growth rate is equal to death rate) often due to a growth-limiting factor such as the depletion of an essential nutrient, and/or the formation of an inhibitory product such as an organic acid. Accordingly, in some embodiments, the bacteria included in a bacterial composition disclosed herein are in early stationary phase. Whether bacteria are in early stationary phase can be determined by any method known in the art. See, e.g., Schorl, C. and Sedivy, J. M., Methods 41(2): 143-150 (2007), which is incorporated by reference in its entirety.

In some embodiments, a bacterial composition disclosed herein results in increased stability of the bacteria when dried, compared to a reference composition (e.g., lacks one of the excipients described herein, e.g., urea). In some embodiments, a bacterial composition provided herein results in increased stability of the bacteria when dried, compared to stability of the bacteria dried in a commercially available freeze-drying formulation, e.g., OPS Diagnostics' Microbial Freeze Drying Buffer (OPS Diagnostics, Lebanon, N.J.). As used herein, the term “stability” refers to the property of being stable (e.g., maintaining viability and/or potency for extended period of time at a specific condition).

In some embodiments, the stability of the bacteria can be assessed by comparing the number of viable bacteria (e.g., colony forming units) at two specific time points and determining the percentage of recovered viable bacteria (i.e., number of viable bacteria at one time point relative to the number of viable bacteria at another time point). For example, a 50% recovery of bacteria indicates that half of the bacteria remained stable over the period of time; and a 100% recovery of bacteria indicates that all (or substantially all) bacteria remained stable over the period of time.

In some embodiments, a bacterial composition disclosed herein results in recovery of at least about 1%, at least about 5%, at least about 10%, at least about 20%, at least about 30%, at least about 40%, at least about 50%, at least about 60%, at least about 70%, at least about 80%, at least about 90% or up to about 100% of the colony forming units of the bacteria over a period of time. In some embodiments, a bacterial composition disclosed herein results in recovery of 1%, 5%, 10%, 20%, 30%, 40%, 50%, 60%, 70%, 80%, 90% or up to 100% of the colony forming units of the bacteria over a period of time. In some embodiments, the period of time is at least about 1 week, at least about 2 weeks, at least about 4 weeks, at least about 2 months, at least about 3 months, at least about 6 months, or at least about 1 year or more, e.g., at 30° C., 4° C., or −20° C.

In some embodiments, a bacterial composition disclosed herein increases the stability of the bacteria when lyophilized, such that the bacteria remain stable over an extended period of time at a defined range of temperature. In certain embodiments, the defined range of temperature comprises about 55° C., about 50° C., about 45° C., about 40° C., about 35° C., about 30° C., about 25° C., about 20° C., about 15° C., about 10° C., about 5° C., about 0° C., about −5° C., about −10° C., about −15° C., about −20° C., about −25° C., about −30° C., about −35° C., about −40° C., about −45° C., about −50° C., about −55° C., about −60° C., or about −65° C. In certain embodiments, the defined range of temperature at which the bacteria remain stable is about −65° C. or lower. In some embodiments, the defined range of temperature comprises 55° C., 50° C., 45° C., 40° C., 35° C., 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., or −65° C. In certain embodiments, the defined range of temperature at which the bacteria remain stable is −65° C. or lower. In certain embodiments, a population of bacteria of a bacterial composition disclosed herein, when lyophilized, remains stable for at least 1 week at 30° C. In some embodiments, a population of bacteria of a bacterial composition disclosed herein, when lyophilized, remains stable for at least 2 weeks at 30° C. In further embodiments, a population of bacteria of a bacterial composition of the present disclosure, when lyophilized remains stable for at least 1 week at 4° C. In other embodiments, a population of bacteria of a bacterial composition disclosed herein, when lyophilized, remains stable for at least 2 weeks at 4° C.

In some embodiments, a bacterial composition of the present disclosure increases the viability of the bacteria, such that there is greater yield after drying, compared to a reference composition (e.g., lacks one of the excipients described herein, e.g., urea). In some embodiments, a bacterial composition provided herein results in increased viability of the bacteria, compared to bacteria dried in a commercially available freeze-dried composition, e.g., OPS Diagnostics' Microbial Freeze Drying Buffer (OPS Diagnostics, Lebanon, N.J.). As used herein, the term “viability” refers to the ability of the bacteria to survive the harsh and stressful conditions involved in a drying process (e.g., lyophilization). Therefore, in certain embodiments, the term “viability” is synonymous with “dry yield” (i.e., the yield or amount of the original viable bacteria recovered after the drying process).

In some embodiments, a bacterial composition disclosed herein increases the viability of the bacteria, such that the dry yield of the bacteria after lyophilization is increased by at least about 1%, about 5%, about 10%, about 20%, about 30%, about 40%, about 50%, about 60%, about 70%, about 80%, about 90%, or up to about 100%, compared to a reference composition (e.g., lacks one of the excipients described herein, e.g., urea). In some embodiments, a bacterial composition disclosed herein increases the viability of the bacteria, such that dry yield of the bacteria after lyophilization is increased by at least 1%, 5%, 10%, 20%, 30%, 40%, 50%, 60%, 70%, 80%, 90%, or up to 100%, compared to a reference composition (e.g., lacks one of the excipients described herein, e.g., urea).

In some embodiments, the stability and/or the viability of the bacteria can be shown as log reduction in the concentration of viable bacteria (CFU/mL). In certain embodiments, the log reduction of the bacteria after drying (i.e., viability) is less than about 1.0, 0.9, 0.8, 0.7, 0.6, 0.5, 0.4, 0.3 0.2, or 0.1. In further embodiments, the log reduction of the bacteria after 1 week at 30° C. (accelerated stability condition) is less than about 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1. In some embodiments, the log reduction of the bacteria after 3 weeks at 30° C. (accelerated stability condition) is less than about 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1. In certain embodiments, the log reduction of the bacteria after 5 weeks at 30° C. (accelerated stability condition) is less than about 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1. In some embodiments, the log reduction of the bacteria after drying is less than 1.0, 0.9, 0.8, 0.7, 0.6, 0.5, 0.4, 0.3, 0.2, or 0.1. In further embodiments, the log reduction of the bacteria after 1 week at 30° C. (accelerated stability condition) is less than 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1. In some embodiments, the log reduction of the bacteria after 3 weeks at 30° C. (accelerated stability condition) is less than 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1. In certain embodiments, the log reduction of the bacteria after 5 weeks at 30° C. (accelerated stability condition) is less than 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1.

In some embodiments, a bacterial composition disclosed herein can additionally include any other pharmaceutically acceptable components known in the art, e.g., diluents, bulking agents, preservatives, salts (e.g., potassium salt, e.g., potassium chloride), binders, compaction agents, lubricants, dispersion enhancers, disintegration agents, flavoring agents, sweeteners, coloring agents, glidants, sorbents, coating agents, vehicles, antioxidants, amino acids, surfactants, buffers, complexation agents, tonicifying agents, polymers, solubilizing agents, and combinations thereof. In some embodiments, a formulation or composition disclosed herein comprises one or more collapse temperature modifiers. Non-limiting examples of collapse temperature modifiers include hydrolyzed gelatin, hydrolyzed collagen, Ficol™, hydroxyethyl starch, Dextran 70, and combinations thereof. See, e.g., Pramanick S., et al., Pharma Times 45(3): 65-77 (2013); Mehmood Y., and Farooq U., Open Science Journal of Pharmacy and Pharmacology 3(3): 19-27 (2015), both of which are hereby incorporated by reference in their entirety.

In some embodiments, a formulation or composition of the present disclosure further comprises a diluent as an excipient. In such embodiments, the excipient can be a solid, semi-solid, or liquid material that acts as a vehicle, carrier, or medium for the active component (e.g., bacteria of the composition disclosed herein). Thus, in some embodiments, a bacterial composition disclosed herein can be in the form of a tablet, capsule, pill, powder, lozenge, sachet, cachet, elixir, suspension, emulsion, solution, syrup, aerosol (as a solid or in a liquid medium), ointment containing, for example, up to 10% by weight of the active component, soft capsule, hard capsule, gel-cap, tablet, suppository, solution, packaged powder, or combinations thereof. In some cases, maximizing delivery of viable bacteria is enhanced by including gastro-resistant polymers, adhesion enhancers, or controlled release enhancers in a formulation, as part of the capsule or as a coating on a tablet, pill or capsule

In some embodiments, a formulation or composition disclosed herein further comprises a preservative. Non-limiting examples of suitable preservatives include antioxidants, such as alpha-tocopherol and ascorbate (ascorbic acid). Accordingly, in certain embodiments, an antioxidant described elsewhere in the present disclosure (e.g., section I) can also function as a preservative. Typically, when used in a composition comprising live bacteria, either no preservative is used or preservative is present in an amount that does not significantly affect viability of the bacteria (e.g., such as concentrations disclosed in the present disclosure).

In some embodiments, a formulation or composition disclosed herein further comprises a binder. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof. As will be apparent to those skilled in the art, in some aspects, excipients disclosed in the present disclosure can have multiple functions in a formulation or composition disclosed herein. For instance, in certain embodiments, certain saccharides (e.g., sugars, e.g., sucrose) can function as a cryoprotectant, a binder, or both.

In some embodiments, a formulation or composition further comprises a lubricant. Non-limiting examples of suitable lubricants include magnesium stearate, glycerol dibehenate, sodium stearyl fumarate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, light mineral oil, and combinations thereof.

In some embodiments, a formulation or composition disclosed herein further comprises a glidant. Non-limiting examples of suitable glidants include fumed silica (colloidal silicon dioxide), talc, magnesium stearate, starch, and combinations thereof.

In some embodiments, a formulation or composition disclosed herein further comprises a dispersion enhancer. Non-limiting examples of suitable dispersants include starch, alginic acid, polyvinylpyrrolidones, guar gum, kaolin, bentonite, purified wood cellulose, sodium starch glycolate, isoamorphous silicate, microcrystalline cellulose, high HLB emulsifier surfactants, and combinations thereof.

In some embodiments, a formulation or composition further comprises a disintegrant. In some embodiments, the disintegrant is a non-effervescent disintegrant. Non-limiting examples of suitable non-effervescent disintegrants include starches, such as corn starch, potato starch, pregelatinized and modified starches thereof, clays, such as bentonite, micro-crystalline cellulose, alginates, sodium starch glycolate, gums, such as agar, guar, locust bean, karaya, pectin, tragacanth, and combinations thereof. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, a formulation or composition of the present disclosure further comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot; and combinations thereof.

In some embodiments, a formulation or composition further comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like; and combinations thereof. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, a formulation or composition disclosed herein further comprises a coloring agent. Non-limiting examples of suitable color agents include food, drug and cosmetic colors (FD&C), drug and cosmetic colors (D&C), external drug and cosmetic colors (Ext. D&C), and combinations thereof. The coloring agents can be used as dyes or their corresponding lakes. Non-limiting examples of dyes include natural dyes, such as beet, radish extracts, and carmine.

Additional suitable excipients that can be included in a formulation or composition disclosed herein include, for example, saline, phosphate buffered saline (PBS), cocoa butter, polyethylene glycol, polyalcohol (e.g., glycerol, sorbitol, or mannitol) and prebiotic oligosaccharides such as inulin, Crystalean® starch, dextrin, and combinations thereof. The additional components can also be selected to account, at least in part, for the ability of the OTUs in a particular composition to withstand gastric pH (if being delivered orally or directly to the GI tract) and/or bile acids, or other conditions encountered by the formulation upon delivery to a subject.

### III. Dry Powder

In some aspects, the present disclosure provides a dry powder (e.g., lyophilisate powder) comprising urea and one or more additional excipients disclosed herein. In some embodiments, the one or more additional excipients comprise a cryoprotectant, an amino acid source, an antioxidant, a salt, a buffering agent, or any combinations thereof. For example, in certain embodiments, a dry powder comprises urea and a cryoprotectant. In other embodiments, a dry powder comprises urea, a cryoprotectant, and an amino acid source. In further embodiments, a dry powder comprises urea, a cryoprotectant, and an antioxidant. In some embodiments, a dry powder comprises urea, a cryoprotectant, and a salt. In other embodiments, a dry powder comprises urea, a cryoprotectant, and a buffer. In certain embodiments, a dry powder comprises urea, a cryoprotectant, an amino acid source, and an antioxidant. In some embodiments, a dry powder comprises urea, a cryoprotectant, an amino acid source, and a salt. In further embodiments, a dry powder comprises urea, a cryoprotectant, an amino acid source, and a buffer. In some embodiments, a dry powder comprises urea, a cryoprotectant, an amino acid source, an antioxidant, and a salt. In certain embodiments, a dry powder comprises urea, a cryoprotectant, an amino acid source, an antioxidant, and a buffer. In some embodiments, a dry powder comprises urea, a cryoprotectant, an amino acid source, an antioxidant, a salt, and a buffer.

In some embodiments, a cryoprotectant is a sugar. In some embodiments, the sugar is a disaccharide, such as sucrose, trehalose, lactose, maltose, cellobiose, chitobiose, or lactulose. In certain embodiments, the disaccharide is sucrose. In other embodiments, the disaccharide is trehalose. Accordingly, in certain embodiments, a dry powder disclosed herein comprises urea and sucrose. In further embodiments, a dry powder comprises urea and trehalose. In some embodiments, a dry powder comprises urea, sucrose, and trehalose.

In some embodiments, an amino acid source is an albumin. In certain embodiments, a dry powder comprises urea, sucrose, and albumin. In other embodiments, a dry powder comprises urea, trehalose, and albumin. In further embodiments, a dry powder comprises urea, sucrose, trehalose, and albumin. In certain embodiments, the albumin is human albumin. In some embodiments, the human albumin is human serum albumin.

In other embodiments, an amino acid source is a gelatin (e.g., hydrolyzed gelatin (e.g., Nutra® or AppliChem®)), a collagen (e.g., hydrolyzed collagen (e.g., VacciPro®)), or a casein (e.g., hydrolyzed casein (e.g. Hy-Case SF)). In some embodiments, a dry powder comprises urea, sucrose, and gelatin. In other embodiments, a dry powder comprises urea, trehalose, and gelatin. In further embodiments, a dry powder comprises urea, sucrose, trehalose, and gelatin. In some embodiments, a dry powder comprises urea, sucrose, and collagen. In other embodiments, a dry powder comprises urea, trehalose, and collagen. In some embodiments, a dry powder comprises urea, sucrose, trehalose, and collagen.

In certain embodiments, a dry powder disclosed herein (e.g., lyophilisate powder) does not comprise albumin (e.g., human albumin), collagen (e.g., hydrolyzed collagen), gelatin (e.g., hydrolyzed gelatin), and/or casein (e.g., hydrolyzed casein).

In some embodiments, an antioxidant is cysteine. In other embodiments, an antioxidant is ascorbic acid. For instance, in some embodiments, a dry powder disclosed herein comprises urea, sucrose, and cysteine. In certain embodiments, a dry powder comprises urea, trehalose, and cysteine. In further embodiments, a dry powder comprises urea, sucrose, trehalose, and cysteine. In some embodiments, a dry powder comprises urea, sucrose, and ascorbic acid. In other embodiments, a dry powder comprises urea, trehalose, and ascorbic acid. In further embodiments, a dry powder comprises urea, sucrose, trehalose, and ascorbic acid.

In some embodiments, a salt comprises a potassium salt. In certain embodiments, the potassium salt is potassium chloride (KCl). For example, in certain embodiments, a dry powder comprises urea, sucrose, and potassium salt. In other embodiments, a dry powder comprises urea, trehalose, and potassium salt. In some embodiments, a dry powder comprises urea, sucrose, trehalose, and potassium salt.

In some embodiments, a buffering agent comprises HEPES. In other embodiments, a buffering agent comprises histidine. In some embodiments, a dry powder disclosed herein comprises urea, sucrose, and HEPES. In other embodiments, a dry powder comprises urea, trehalose, and HEPES. In further embodiments, a dry powder comprises urea, sucrose, trehalose, and HEPES. In some embodiments, a dry powder disclosed herein comprises urea, sucrose, and histidine. In further embodiments, a dry powder disclosed herein comprises urea, trehalose, and histidine. In still further embodiments, a dry powder disclosed herein comprises urea, sucrose, trehalose, and histidine

In some embodiments, a dry powder disclosed herein comprises two components described above (e.g., urea and a cryoprotectant). In other embodiments, a dry powder comprises three components described above. In some embodiments, a dry powder comprises four components described above. In further embodiments, a dry powder comprises five components described above. In certain embodiments, a dry powder comprises six components described above. In still further embodiments, a dry powder disclosed herein comprises seven components described above. In some embodiments, a dry powder comprises eight components described above. In certain embodiments, a dry powder comprises nine components described above. In other embodiments, a dry powder comprises ten components described above.

In certain embodiments, a dry powder of the present disclosure (e.g., lyophilisate powder) has a density of about 0.1 g/cm³, about 0.2 g/cm³, about 0.3 g/cm³, about 0.4 g/cm³, about 0.5 g/cm³, about 0.6 g/cm³, about 0.7 g/cm³, about 0.8 g/cm³, about 0.9 g/cm³, or about 1.0 g/cm³. In some embodiments, a dry powder of the present disclosure (e.g., lyophilisate powder) has a density of about 0.3 g/cm³. In certain embodiments, a dry powder of the present disclosure (e.g., lyophilisate powder) has a density of about 0.4 g/cm³. In certain embodiments, a dry powder of the present disclosure (e.g., lyophilisate powder) has a density of 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, or 1.0 g/cm³. In some embodiments, a dry powder of the present disclosure (e.g., lyophilisate powder) has a density of 0.3 g/cm³. In certain embodiments, a dry powder of the present disclosure (e.g., lyophilisate powder) has a density of 0.4 g/cm³.

In some embodiments, a dry powder (e.g., lyophilisate powder) has a particle size distribution of about 1 μm, about 5 μm, about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm. In certain embodiments, a dry powder disclosed herein (e.g., lyophilisate powder) has a particle size distribution of about 10 μm to about 150 μm, such as about 10 μm, about 45 μm, or about 140 μm. In some embodiments, a dry powder (e.g., lyophilisate powder) has a particle size distribution of 1 μm, 5 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, or 300 μm. In certain embodiments, a dry powder disclosed herein (e.g., lyophilisate powder) has a particle size distribution of 10 μm to 150 μm, such as 10 μm, 45 μm, or 140 μm.

In some embodiments, a dry powder of the present disclosure (e.g., lyophilisate powder) further comprises one or more bacteria (e.g., one or more different OTUs of viable bacteria). Accordingly, in certain embodiments, a dry powder disclosed herein (e.g., lyophilisate powder) comprises (a) one or more bacteria (e.g., those disclosed herein), (b) urea, sucrose, hydrolyzed collagen, KCl, and/or HEPES. In some embodiments, a dry powder comprises one or more bacteria, urea, sucrose, and gelatin hydrolysate.

In some embodiments, a dry powder of the present disclosure (e.g., lyophilisate powder) comprises a single bacterium. In other embodiments, a dry powder (e.g., lyophilisate powder) comprises 2 or more types of bacteria. Accordingly, in certain embodiments, a dry powder disclosed herein (e.g., lyophilisate powder) comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 50, or greater than 50 types of bacteria, as defined by species or operational taxonomic unit (OTU).

In some embodiments, a dry powder of the present disclosure (e.g., lyophilisate powder) comprises two or more different dry powders, wherein each of the different dry powders comprises one or more different types of bacteria. In certain embodiments, a dry powder comprises two different dry powders, wherein each different dry powder comprises a different type of bacteria. In some embodiments, a dry powder comprises three different dry powders, wherein each different dry powder comprises a different type of bacteria. In further embodiments, a dry powder comprises four different dry powders, wherein each different dry powder comprises a different type of bacteria. In certain embodiments, a dry powder comprises five or more different dry powders, wherein each different dry powder comprises a different type of bacteria.

In some embodiments, the bacteria can be present in approximately equal amounts of viable bacteria from each family, genus, species, or OTU (e.g., those described supra). In other embodiments, the bacteria are present in varying amounts in the dry powder (e.g., lyophilisate powder). For example, in a dry powder with two types of bacteria, the bacteria can be present in from a 1:10,000 ratio to a 1:1 ratio, from a 1:10,000 ratio to a 1:1,000 ratio, from a 1:1,000 ratio to a 1:100 ratio, from a 1:100 ratio to a 1:50 ratio, from a 1:50 ratio to a 1:20 ratio, from a 1:20 ratio to a 1:10 ratio, from a 1:10 ratio to a 1:1 ratio. For dry powder comprising at least three types of bacteria, the ratio of type of bacteria can be chosen pairwise from ratios for dry powder with two types of bacteria. For example, in a dry powder comprising bacteria A, B, and C, at least one of the ratio between bacteria A and B, the ratio between bacteria B and C, and the ratio between bacteria A and C can be chosen, independently, from the pairwise combinations above

In some embodiments, a dry powder disclosed herein (e.g., lyophilisate powder) comprises anaerobic bacteria. In some embodiments, a dry powder provided herein comprises (i) one or more anaerobic bacteria, (ii) urea, (iii) cryoprotectant, and (iv) an amino acid source. In some embodiments, a dry powder comprises (i) one or more anaerobic bacteria, (ii) urea, (iii) sucrose, and (iv) gelatin hydrolysate. In some embodiments, a dry powder comprises (i) one or more anaerobic bacteria, (ii) about 0.5% urea, (iii) about 10% sucrose, and (iv) about 3% gelatin hydrolysate.

In certain embodiments, the anaerobic bacteria are obligate anaerobes. In some embodiments, a dry powder provided herein comprises (i) one or more obligate anaerobes, (ii) urea, (iii) cryoprotectant, and (iv) an amino acid source. In some embodiments, a dry powder comprises (i) one or more obligate anaerobes, (ii) urea, (iii) sucrose, and (iv) gelatin hydrolysate. In some embodiments, a dry powder comprises (i) one or more obligate anaerobes, (ii) about 0.5% urea, (iii) about 10% sucrose, and (iv) about 3% gelatin hydrolysate.

In other embodiments, the anaerobic bacteria are facultative anaerobes. In some embodiments, a dry powder provided herein comprises (i) one or more facultative anaerobes, (ii) urea, (iii) cryoprotectant, and (iv) an amino acid source. In some embodiments, a dry powder comprises (i) one or more facultative anaerobes, (ii) urea, (iii) sucrose, and (iv) gelatin hydrolysate. In some embodiments, a dry powder comprises (i) one or more facultative anaerobes, (ii) about 0.5% urea, (iii) about 10% sucrose, and (iv) about 3% gelatin hydrolysate.

In other embodiments, the anaerobic bacteria are aerotolerant anaerobes. In some embodiments, a dry powder provided herein comprises (i) one or more aerotolerant anaerobes, (ii) urea, (iii) cryoprotectant, and (iv) an amino acid source. In some embodiments, a dry powder comprises (i) one or more aerotolerant anaerobes, (ii) urea, (iii) sucrose, and (iv) gelatin hydrolysate. In some embodiments, a dry powder comprises (i) one or more aerotolerant anaerobes, (ii) about 0.5% urea, (iii) about 10% sucrose, and (iv) about 3% gelatin hydrolysate.

In some embodiments, a dry powder disclosed herein (e.g., lyophilisate powder) comprises aerobes. In some embodiments, a bacterial composition provided herein comprises (i) one or more aerobes, (ii) urea, (iii) cryoprotectant, and (iv) an amino acid source. In some embodiments, a composition comprises (i) one or more aerobes, (ii) urea, (iii) sucrose, and (iv) gelatin hydrolysate. In some embodiments, a composition comprises (i) one or more aerobes, (ii) about 0.5% urea, (iii) about 10% sucrose, and (iv) about 3% gelatin hydrolysate.

In certain embodiments, a dry powder of the present disclosure (e.g., lyophilisate powder) comprises at least one anaerobe (e.g., aerotolerant anaerobes) and at least one aerobe. In some embodiments, a dry powder provided herein comprises (i) one or more anaerobes and one or more aerobes, (ii) urea, (iii) cryoprotectant, and (iv) an amino acid source. In some embodiments, a dry powder comprises (i) one or more anaerobes and one or more aerobes, (ii) urea, (iii) sucrose, and (iv) gelatin hydrolysate. In some embodiments, a dry powder comprises (i) one or more anaerobes and one or more aerobes, (ii) about 0.5% urea, (iii) about 10% sucrose, and (iv) about 3% gelatin hydrolysate.

In some embodiments, anaerobic bacteria, when present in a dry powder disclosed herein (e.g., lyophilisate powder), have increased aerotolerance (e.g., remains stable for at least 3 hours post-lyophilization in the presence of oxygen) compared to corresponding anaerobic bacteria in a reference dry powder (e.g., lacks one of the excipients described herein, e.g., urea).

In some embodiments, a dry powder disclosed herein (e.g., lyophilisate powder) comprises one or more bacteria from a family, genus, species, or OTU useful in treating a subject with a microbiome-related disease or disorder. The subject may have a dysbiosis, e.g., of the GI tract, an infection, be at risk for infection (for example, infection associated with antibiotic treatment, radiation, chemotherapy), or have another disease or disorder affected by the microbiome (for example, an inflammatory bowel disease, obesity, diabetes, asthma/allergy, an autoimmune disease, a central nervous system (CNS) disease or disorder (e.g., Autism Spectral Disorder (ASD) or Parkinson's Disease (PD)), a cholestatic disease, gastric ulcers, chronic heart diseases, rheumatic diseases, kidney diseases, or cancer). In certain embodiments, a dry powder disclosed herein (e.g., lyophilisate powder) comprises one or more bacteria that are present with high prevalence and/or high abundance in healthy individuals compared to an individual having a disease or risk factor.

In some embodiments, a dry powder of the present disclosure (e.g., lyophilisate powder) comprises one or more commensal bacteria derived from a human. In some embodiments, one or more bacteria in the dry powder described herein are Firmicutes. In some embodiments, the dry powder comprises bacteria from the class Clostridia. In some embodiments, the dry powder comprises bacteria from the order Clostridiales. In some embodiments, the dry powder comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In certain embodiments, a dry powder (e.g., lyophilisate powder) can comprise at least one, two, three, four, five, six, seven, eight, nine, ten, eleven, twelve, thirteen, fourteen, or all of the families listed. For example, in some embodiments, a dry powder comprises bacteria from one of the families listed above. In other embodiments, a dry powder comprises bacteria from two of the families listed above. In further embodiments, a dry powder comprises bacteria from three of the families listed above. In some embodiments, a dry powder comprises bacteria from four of the families listed above. In certain embodiments, a dry powder comprises bacteria from five of the families listed above. In further embodiments, a dry powder comprises bacteria from six of the families listed above. In still further embodiments, a dry powder comprises bacteria from seven of the families listed above. In some embodiments, a dry powder comprises bacteria from eight of the families listed above. In some embodiments, a dry powder comprises bacteria from nine of the families listed above. In some embodiments, a dry powder comprises bacteria from ten of the families listed above. In some embodiments, a dry powder comprises bacteria from eleven of the families listed above. In some embodiments, a dry powder comprises bacteria from twelve of the families listed above. In some embodiments, a dry powder comprises bacteria from thirteen of the families listed above. In some embodiments, a dry powder comprises bacteria from fourteen of the families listed above. In some embodiments, a dry powder comprises bacteria from fifteen of the families listed above.

In some embodiments, a dry powder disclosed herein comprises (i) bacteria from one or more of the families listed above, (ii) urea, and (iii) a cryoprotectant. In other embodiments, a dry powder comprises (i) bacteria from one or more of the families listed above, (ii) urea, (iii) a cryoprotectant, and (iv) an amino acid source. In further embodiments, a dry powder comprises (i) bacteria from one or more of the families listed above, (ii) urea, (iii) a cryoprotectant, (iv) and an antioxidant. In some embodiments, a dry powder comprises (i) bacteria from one or more of the families listed above, (ii) urea, (iii) a cryoprotectant, and (iv) a salt. In other embodiments, a dry powder comprises (i) bacteria from one or more of the families listed above, (ii) urea, (iii) a cryoprotectant, and (iv) a buffer. In certain embodiments, a dry powder comprises (i) bacteria from one or more of the families listed above, (ii) urea, (iii) a cryoprotectant, (iv) an amino acid source, and (v) an antioxidant. In some embodiments, a dry powder comprises (i) bacteria from one or more of the families listed above, (ii) urea, (iii) a cryoprotectant, (iv) an amino acid source, and (v) a salt. In further embodiments, a dry powder comprises (i) bacteria from one or more of the families listed above, (ii) urea, (iii) a cryoprotectant, (iv) an amino acid source, and (v) a buffer. In some embodiments, a dry powder comprises (i) bacteria from one or more of the families listed above, (ii) urea, (iii) a cryoprotectant, (iv) an amino acid source, (v) an antioxidant, and (vi) a salt. In certain embodiments, a dry powder comprises (i) bacteria from one or more of the families listed above, (ii) urea, (iii) a cryoprotectant, (iv) an amino acid source, (v) an antioxidant, and a (vi) buffer. In some embodiments, a dry powder comprises (i) bacteria from one or more of the families listed above, (ii) urea, (iii) a cryoprotectant, (iv) an amino acid source, (v) an antioxidant, (vi) a salt, and (vii) a buffer.

In some embodiments, a dry powder disclosed herein (e.g., lyophilisate powder) comprises a population of bacteria that has been purified from a biological material (e.g., fecal material, such as feces or materials isolated from the various segments of the small and/or large intestine) obtained from a mammalian donor subject (e.g., a healthy human or a human responsive to a treatment, such as an immuno-oncology treatment). In some embodiments, the biological material (e.g., fecal material) is obtained from multiple donors (e.g., 2, 3, 4, 5, 6, 7, 8, 9, 10, 15, 20, 25, 30, 35, 40, 45, 50, 75, 100, 200, 300, 400, 500, 750, 1000, or from greater than 1000 donors), and the materials are pooled prior to purification or after purification of the desired bacteria. In other embodiments, the biological material (sample) can be obtained from a single donor subject at multiple times and two or more samples pooled, e.g., 2, 3, 4, 5, 6, 7, 8, 9, 10, 15, 20, 25, 30, 32, 35, 45, 48, 50, 100 samples from a single donor. Methods of making such preparations include treatment of the feces with chloroform, acetone, ethanol, and the like, e.g., see PCT/US2014/014745 and U.S. Pat. No. 9,011,834, which are incorporated herein by reference in their entirety.

In some embodiments, a dry powder (e.g., lyophilisate powder) comprises both spore-forming bacteria and non-spore-forming bacteria. In certain embodiments, the non-spore-forming bacteria are Gram-negative bacteria (e.g., Bacteroides faecis or Bacteroides sp_4_1_36). In some embodiments, the spore-forming bacteria are Gram-positive bacteria (e.g., Clostridium bolteae or Clostridium sp_D5). In certain embodiments, a dry powder of the present disclosure (e.g., lyophilisate powder) comprises only spore-forming bacteria. In some embodiments, the spore-forming bacteria are all in the form of spores. In other embodiments, some of the spore-forming bacteria are in the form of spores, while other spore-forming bacteria are in a vegetative form. Non-limiting examples of other bacterial strains that can be included in a bacterial composition of the present disclosure include those listed in Table 4, Table 5, FIG. 13, FIG. 17, FIG. 30, FIG. 31, or FIG. 32 of International Publication No. WO 2019/227085 A1, which is herein incorporated by reference in its entirety.

In some embodiments, a dry powder disclosed herein comprises (i) a population of bacteria, (ii) urea, (iii) a cryoprotectant, and (iv) an amino acid source, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In some embodiments, a dry powder disclosed herein comprises (i) a population of bacteria, (ii) urea, (iii) sucrose, and (iv) gelatin hydrolysate, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In some embodiments, a dry powder disclosed herein comprises (i) a population of bacteria, (ii) about 0.5% urea, (iii) about 10% sucrose, and (iv) about 3% gelatin hydrolysate, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae.

In some embodiments, a dry powder disclosed herein comprises (i) a population of bacteria, (ii) urea, (iii) a cryoprotectant, (iv) an amino acid source, (v) an antioxidant, (vi) a salt, and (vii) a buffering agent, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In certain embodiments, a dry powder comprises (i) a population of bacteria, (ii) urea, (iii) sucrose, (iv) gelatin hydrolysate, (v) ascorbic acid, (vi) potassium chloride, and (vii) HEPES, wherein the population of bacteria comprise bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae. In further embodiments, a dry powder comprises (i) a population of bacteria, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES, wherein the population of bacteria comprises bacteria from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae.

In some embodiments, a dry powder comprises bacteria having a 16S rDNA sequence that is at least 97% identical to the 16S rDNA sequence set forth in SEQ ID NOs: 1 to 398. Accordingly, in certain embodiments, a dry powder disclosed herein comprises (i) one or more bacteria comprising a 16S rDNA sequence that is at least 97% identical to the 16S rDNA sequence set forth in SEQ ID NOs: 1 to 398, (ii) urea, (iii) a cryoprotectant, and (iv) an amino acid source. In some embodiments, a dry powder comprises (i) one or more bacteria comprising a 16S rDNA sequence that is at least 97% identical to the 16S rDNA sequence set forth in SEQ ID NOs: 1 to 398, (ii) urea, (iii) sucrose, and (iv) gelatin hydrolysate. In further embodiments, a dry powder comprises (i) one or more bacteria comprising a 16S rDNA sequence that is at least 97% identical to the 16S rDNA sequence set forth in SEQ ID NOs: 1 to 398, (ii) about 0.5% urea, (iii) about 10% sucrose, and (iv) about 3% gelatin hydrolysate.

In some embodiments, a dry powder disclosed herein comprises (i) one or more bacteria comprising a 16S rDNA sequence that is at least 97% identical to the 16S rDNA sequence set forth in SEQ ID NOs: 1 to 398, (ii) urea, (iii) a cryoprotectant, (iv) an amino acid source, (v) an antioxidant, (vi) a salt, and (vii) a buffering agent. In some embodiments, a dry powder comprises (i) one or more bacteria comprising a 16S rDNA sequence that is at least 97% identical to the 16S rDNA sequence set forth in SEQ ID NOs: 1 to 398, (ii) urea, (iii) sucrose, (iv) gelatin hydrolysate, (v) ascorbic acid, (vi) potassium chloride, and (vii) HEPES. In further embodiments, a dry powder comprises (i) one or more bacteria comprising a 16S sequence that is at least 97% identical to the 16S rDNA sequence set forth in SEQ ID NOs: 1 to 398, (ii) about 0.5% urea, (iii) about 10% sucrose, (iv) about 3% gelatin hydrolysate, (v) about 1% ascorbic acid, (vi) about 25 mM potassium chloride, and (vii) about 50 mM HEPES.

In some embodiments, a dry powder disclosed herein (e.g., lyophilisate powder) is encapsulated. A capsule typically comprises a core material comprising an active component (e.g., a lyophilisate powder disclosed herein) and a shell wall that encapsulates the core material. In some embodiments, the shell wall material comprises at least one of a soft gelatin, a hard gelatin, or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; shellac (purified lac); acetate succinate (HPMC-AS, e.g., VCAPS® enteric and intrinsic capsules); and combinations thereof. In some embodiments, at least one polymer functions as taste-masking agents. In certain embodiments, the shell wall of the capsule is enterically-coated, such that the capsule can resist disintegration in the stomach and permits the core material (e.g., a dry powder disclosed herein, e.g., a lyophilisate powder) to pass intact into the duodenum or to be delayed in release.

In some embodiments, a dry powder of the present disclosure (e.g., lyophilisate powder) is reconstituted by a reconstitution solution. Non-limiting examples of reconstitution solutions are known in the art and include water, physiologic solutions (e.g. saline, lactated ringers), and any pharmaceutically acceptable buffer (e.g., in humans).

In some embodiments, a dry powder disclosed herein (e.g., lyophilisate powder) can be incorporated into a food product. In some embodiments, the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula, and combinations thereof. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments, the food product is a solid foodstuff. Suitable examples of a solid foodstuff include without limitation a food bar, a snack bar, a cookie, a brownie, a muffin, a cracker, an ice cream bar, a frozen yogurt bar, and combinations thereof.

In some embodiments, a dry powder disclosed herein (e.g., lyophilisate powder) is incorporated into a therapeutic food. In some embodiments, the therapeutic food is a ready-to-use food that optionally contains some or all essential macronutrients and micronutrients. In some embodiments, a dry powder disclosed herein (e.g., lyophilisate powder) is incorporated into a supplementary food that is designed to be blended into an existing meal. In some embodiments, the supplemental food contains some or all essential macronutrients and micronutrients. In some embodiments, a dry powder disclosed herein (e.g., lyophilisate powder) is blended with or added to an existing food to fortify the food's protein nutrition. Examples include food staples (grain, salt, sugar, cooking oil, margarine), beverages (coffee, tea, soda, beer, liquor, sports drinks), snacks, sweets, and other foods.

### IV. Methods of Drying Bacteria

Methods of drying (e.g., lyophilizing) compositions, including compositions comprising bacteria, are known in the art. See, e.g., U.S. Pat. Nos. 3,261,761; 4,205,132; 4,518,696; PCT Publication Nos: WO 2014/029578; WO 2012/098358; WO 2012/076665; WO 2016/083617; and WO 2012/088261, which are incorporated herein by reference in their entirety. However, finding conditions that allow for the drying of bacteria, particularly anaerobic bacteria, has been challenging. See, e.g., Peiren, J., et al., Appl Microbiol Biotechnol 99(8): 3559-71 (2015), which is incorporated by reference in its entirety. In some aspects, the present disclosure provides methods of drying one or more bacteria, wherein the dried bacteria have much greater stability, e.g., compared to bacteria dried by other methods known in the art, such as described in the above-cited references. In certain aspects, the present disclosure provides methods of producing a dry powder disclosed herein (e.g., lyophilisate powder). In some aspects, the invention includes bacteria prepared by drying in a formulation disclosed herein.

In some embodiments, bacteria (e.g., aerobic and anaerobic) can be dried using any suitable drying methods known in the art. Non-limiting examples of suitable drying methods include freeze-drying (i.e., lyophilization), spray drying, spray-freeze drying, electrostatic spray drying, or combinations thereof. See, e.g., U.S. Pat. Nos. 6,010,725; 7,007,406; and U.S. Publication No. 2017/0259185, each of which is herein incorporated by reference in its entirety. In certain embodiments, bacterial compositions disclosed herein are dried using lyophilization.

In some embodiments, a method of drying one or more bacteria disclosed herein comprises: (a) freezing a bacterial formulation (e.g., those disclosed herein) (“freezing step”); (b) reducing the pressure of the frozen bacterial formulation by an amount effective to remove any aqueous solvent (e.g., water) from the frozen bacterial formulation (“vacuum step”), and (c) increasing the temperature of the frozen bacterial formulation (“drying step”), thereby producing a dry powder (e.g., lyophilisate powder).

In some embodiments, the density of a dry powder (e.g., lyophilisate powder) produced by the methods disclosed herein is about 0.1 g/cm³, about 0.2 g/cm³, about 0.3 g/cm³, about 0.4 g/cm³, about 0.5 g/cm³, about 0.6 g/cm³, about 0.7 g/cm³, about 0.8 g/cm³, about 0.9 g/cm³, or about 1.0 g/cm³. In some embodiments, a dry powder (e.g., lyophilisate powder) has a density of about 0.3 g/cm³. In certain embodiments, a dry powder (e.g., lyophilisate powder) has a density of about 0.4 g/cm³. In some embodiments, the density of a dry powder (e.g., lyophilisate powder) produced by the methods disclosed herein is 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, or 1.0 g/cm³. In some embodiments, a dry powder (e.g., lyophilisate powder) has a density of 0.3 g/cm³. In certain embodiments, a dry powder has a density of 0.4 g/cm³.

In some embodiments, the particle size distribution of a dry powder (e.g., lyophilisate powder) produced by the present methods is about 1 μm, about 5 μm, about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm. In certain embodiments, a dry powder disclosed herein (e.g., lyophilisate powder) has a particle size distribution of about 10 to 150 μm, such as about 10 μm, about 45 μm, or about 140 μm. In some embodiments, the particle size distribution of a dry powder (e.g., lyophilisate powder) produced by the present methods is 1 μm, 5 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, or 300 μm. In certain embodiments, a dry powder disclosed herein (e.g., lyophilisate powder) has a particle size distribution of 10 to 150 μm, such as 10 μm, 45 μm, or 140 μm.

In some embodiments, the residual moisture in a dry powder (e.g., lyophilisate powder) produced by the methods disclosed herein is less than about 5.0%, about 4.0%, about 3.0%, about 2.0%, about 1.0%, about 0.9%, about 0.8%, about 0.7%, about 0.6%, about 0.5%, about 0.4%, about 0.3%, about 0.2%, or about 0.1%. In some embodiments, the residual moisture is less than about 5%. In other embodiments, the residual moisture is less than about 4%. In certain embodiments, the residual moisture is less than about 3%. In some embodiments, the residual moisture is less than about 2%. In some embodiments, the residual moisture in a dry powder (e.g., lyophilisate powder) produced by the methods disclosed herein is less than 5.0%, 4.0%, 3.0%, 2.0%, 1.0%, 0.9%, 0.8%, 0.7%, 0.6%, 0.5%, 0.4%, 0.3%, 0.2%, or 0.1%. In some embodiments, the residual moisture is less than 5%. In other embodiments, the residual moisture is less than 4%. In certain embodiments, the residual moisture is less than 3%. In some embodiments, the residual moisture is less than 2%.

In some embodiments, when the drying process involves a freezing step, a bacterial formulation is frozen to a freezing temperature of about −65° C. to about −40° C., about −65° C. to about −45° C., about −65° C. to about −55° C., about −60° C. to about −40° C., about −60° C. to about −50° C. or about −60° C. to about −55° C. during the freezing step. In certain embodiments, a bacterial formulation is frozen to a freezing temperature of −65° C. to −40° C., −65° C. to −45° C., −65° C. to −55° C., −60° C. to −40° C., −60° C. to −50° C. or −60° C. to −55° C. during the freezing step. In some embodiments, a bacterial formulation is frozen to the freezing temperature at a temperature rate of about 0.5° C./min, about 0.6° C./min, about 0.7° C./min, about 0.8° C./min, about 0.9° C./min, about 1.0° C./min, about 1.1° C./min, about 1.2° C./min, about 1.3° C./min, about 1.4° C./min, about 1.5° C./min, about 1.6° C./min, about 1.7° C./min, about 1.8° C./min, about 1.9° C./min, about 2.0° C./min, about 2.1° C./min, about 2.2° C./min, about 2.3° C./min, about 2.4° C./min, about 2.5° C./min, about 2.6° C./min, about 2.7° C./min, about 2.8° C./min, about 2.9° C./min, or about 3.0° C./min. In certain embodiments, a bacterial formulation is frozen to the freezing temperature at a temperature rate of 0.5° C./min, 0.6° C./min, 0.7° C./min, 0.8° C./min, 0.9° C./min, 1.0° C./min, 1.1° C./min, 1.2° C./min, 1.3° C./min, 1.4° C./min, 1.5° C./min, 1.6° C./min, 1.7° C./min, 1.8° C./min, 1.9° C./min, 2.0° C./min, 2.1° C./min, 2.2° C./min, 2.3° C./min, 2.4° C./min, 2.5° C./min, 2.6° C./min, 2.7° C./min, 2.8° C./min, 2.9° C./min, or 3.0° C./min. In some embodiments, a bacterial formulation is frozen to a freezing temperature of about −45° C. at a temperature rate of about 1.0° C./min. In certain embodiments, a bacterial formulation is frozen to a freezing temperature of −45° C. at a temperature rate of 1.0° C./min.

In some embodiments, the freezing temperature is held from about 30 minutes to about 7 hours, about 1 hour to about 7 hours, about 1.5 hours to about 7 hours, about 1.5 hours to about 6 hours, about 1.5 hours to about 5 hours, about 1.5 hours to about 4 hours, about 1.5 hours to about 3 hours, or about 1.5 hours to about 2.5 hours during the freezing step. In certain embodiments, the freezing temperature is held from 30 minutes to 7 hours, 1 hour to 7 hours, 1.5 hours to 7 hours, 1.5 hours to 6 hours, 1.5 hours to 5 hours, 1.5 hours to 4 hours, 1.5 hours to 3 hours, or 1.5 hours to 2.5 hours during the freezing step. In some embodiments, the freezing temperature is held from about 4 hours to about 6 hours. In certain embodiments, the freezing temperature is held from 4 hours to 6 hours.

In some embodiments, the drying process disclosed herein comprises a “vacuum step,” which comprises subjecting the frozen bacterial formulation to a vacuum between about 0.05 and about 1 mbar, between about 0.05 and about 0.50 mbar, between about 0.10 and about 0.50 mbar, between about 0.15 and about 0.50 mbar, between about 0.20 and about 0.50 mbar, or between about 0.25 and about 0.50 mbar. In certain embodiments, the “vacuum step” comprises subjecting a frozen bacterial formulation to a vacuum between 0.05 and 1 mbar, between 0.05 and 0.50 mbar, between 0.10 and 0.50 mbar, between 0.15 and 0.50 mbar, between 0.20 and 0.50 mbar, or between 0.25 and 0.50 mbar. In some embodiments, the “vacuum step” comprises subjecting a frozen bacterial formulation to a vacuum between about 0.06 and about 0.2 mbar. In certain embodiments, the “vacuum step” comprises subjecting a frozen bacterial formulation to a vacuum between 0.06 and 0.2 mbar. The unit of mbar can be converted to Torr or any other units. For example, 1 mbar can be converted to 0.75006375541921 Torr. In some embodiments, the vacuum is held in the “vacuum step” for about 5 hours, about 4 hours, about 3 hours, about 2 hours, or about 1 hour. In certain embodiments, the vacuum is held in the “vacuum step” for 5 hours, 4 hours, 3 hours, 2 hours, or 1 hour.

In some embodiments, when a drying process herein comprises a drying step after the freezing and/or vacuum step, the “drying step” (or “primary drying step” where multiple drying steps are used) comprises ramping up the temperature of the frozen bacterial formulation from the freezing temperature (e.g., about −45° C.) to a drying temperature of at least about −30° C. In certain embodiments, the drying temperature is at least about −32° C., at least about −33° C., at least about −34° C., at least about −35° C., at least about −36° C., at least about −37° C., at least about −38° C., at least about −39° C., or at least about −40° C. In some embodiments, the drying temperature is about −34° C. In certain embodiments, the “drying step” comprises ramping up the temperature of the frozen bacterial formulation from the freezing temperature to a drying temperature of at least −30° C. In certain embodiments, the drying temperature is at least −32° C., at least −33° C., at least −34° C., at least −35° C., at least −36° C., at least −37° C., at least −38° C., at least −39° C., or at least −40° C. In other embodiments, the drying temperature is −34° C.

In some embodiments, a drying method disclosed herein comprises a secondary drying step. In certain embodiments, the secondary drying step comprises further ramping up the temperature after the primary drying step to a temperature of at least about 10° C. In some embodiments, the temperature of the secondary drying step is at least about 15° C., at least about 20° C., at least about 25° C., or at least about 30° C. In certain embodiments, the temperature of the secondary drying step is about 20° C. In some embodiments, the temperature of the secondary drying step is at least 15° C., at least 20° C., at least 25° C., or at least 30° C. In certain embodiments, the temperature of the secondary drying step is 20° C.

In some embodiments, the temperature of the bacterial formulation is ramped up to the secondary drying temperature from the primary drying temperature at a rate of about 0.05° C./min, about 0.1° C./min, about 0.15° C./min, about 0.2° C./min, about 0.25° C./min, about 0.3° C./min, about 0.35° C./min, about 0.4° C./min, about 0.45° C./min, about 0.5° C./min, about 0.6° C./min, about 0.7° C./min, about 0.8° C./min, about 0.9° C./min, or about 1.0° C./min. In certain embodiments, the temperature of the bacterial formulation is ramped up to the secondary drying temperature from the primary drying temperature at a rate of 0.05° C./min, 0.1° C./min, 0.15° C./min, 0.2° C./min, 0.25° C./min, 0.3° C./min, 0.35° C./min, 0.4° C./min, 0.45° C./min, 0.5° C./min, 0.6° C./min, 0.7° C./min, 0.8° C./min, 0.9° C./min, or 1.0° C./min. In some embodiments, the temperature of the bacterial formulation is ramped up to the secondary drying temperature from the primary drying temperature at a rate of about 0.1° C./min. In certain embodiments, the temperature of the bacterial formulation is ramped up to the secondary drying temperature from the primary drying temperature at a rate of 0.1° C./min.

In some embodiments, a drying method of the present disclosure comprises holding the temperature of the bacterial formulation at the secondary drying temperature at a pressure of about 0.01 mbar, about 0.02 mbar, about 0.03 mbar, about 0.04 mbar, about 0.05 mbar, about 0.06 mbar, about 0.07 mbar, about 0.08 mbar, about 0.09 mbar, or about 0.1 mbar. In certain embodiments, the secondary drying temperature is held at a pressure of 0.01 mbar, 0.02 mbar, 0.03 mbar, 0.04 mbar, 0.05 mbar, 0.06 mbar, 0.07 mbar, 0.08 mbar, 0.09 mbar, or 0.1 mbar. In some embodiments, the secondary drying temperature is held at a pressure of between 0.06 and 0.07 mbar.

In some embodiments, a method of drying one or more bacteria disclosed herein comprises transferring a bacterial composition (e.g., those disclosed herein) into a container, such as a tube, a bag, a bottle, a tray, a vial (e.g., a glass vial), a syringe, or any other suitable containers, prior to the freezing step, such that the entire drying process (i.e., freezing step, vacuum step, and the drying step) are performed in the container. In certain embodiments, the container is disposable.

In some embodiments, the container is a tray. In certain embodiments, a tray that can be used with the drying methods disclosed herein comprise a steel tray (e.g., stainless steel tray), an aluminum tray, or a plastic tray. In some embodiments, the tray is not a steel tray. In certain embodiments, the tray can be coated with a non-adhesive coating, such as Teflon®. In some embodiments, a tray that can be used with the methods disclosed herein comprises a LyoGuard® tray (W. L. Gore).

In some embodiments, a bacterial composition is transferred to a tray at a solution depth of about 5 cm, about 4 cm, about 3 cm, about 2 cm, about 1 cm, about 0.9 cm, about 0.8 cm, about 0.7 cm, about 0.6 cm, about 0.5 cm, about 0.4 cm, about 0.3 cm, about 0.2 cm, about 0.1 cm or less. In certain embodiments, a solution depth is about 2 cm. In other embodiments, a solution depth is about 1 cm. In further embodiments, a solution depth is about 0.5 cm. In some embodiments, a solution depth is about 0.25 cm. In certain embodiments, a bacterial composition is transferred to a tray at a solution depth of 5 cm, 4 cm, 3 cm, 2 cm, 1 cm, 0.9 cm, 0.8 cm, 0.7 cm, 0.6 cm, 0.5 cm, 0.4 cm, 0.3 cm, 0.2 cm, 0.1 cm or less. In certain embodiments, a solution depth is 2 cm. In other embodiments, a solution depth is 1 cm. In further embodiments, a solution depth is 0.5 cm. In some embodiments, a solution depth is 0.25 cm.

Dried cakes produced by the methods disclosed herein can be assessed based on product quality analysis, reconstitution time, quality of reconstitution, high molecular weight, moisture content, glass transition temperature (Tg), and biological or biochemical activity (e.g. colony forming units (CFU)). Typically, product quality analysis includes product degradation rate analysis using methods including, but not limited to, size exclusion chromatography (SEC), cation exchange-HPLC (CEX-HPLC), X-ray diffraction (XRD), modulated differential scanning calorimetry (mDSC), reversed phase HPLC (RP-HPLC), multi-angle light scattering detector (MALS), fluorescence, ultraviolet absorption, nephelometry, capillary electrophoresis (CE), SDS-PAGE, and combinations thereof. In some embodiments, evaluation of dried product in accordance with the present invention includes a step of evaluating cake appearance. Additionally, dried cakes can be assessed based on biological or biochemical activities of the product.

In some embodiments, a dried cake produced by the methods disclosed herein is not collapsed. As used herein, the term “collapse” refers to loss of an intact structure or change of the original structure of dried cake. Collapse in the product during drying can be detected by various instruments including, but not limited to, product temperature measurement devices, freeze drying microscopy, or instruments detecting electrical resistance. Collapse in dried product (e.g., cake) can be detected manually by visual inspection, residual moisture, Differential Scanning calorimetry (DSC), or BET surface area.

### V. Therapeutic Formulations

The dry powders disclosed herein (e.g., lyophilisate powder) can be used to treat various diseases or disorders (e.g., those associated with a dysbiosis of the gastrointestinal tract). Accordingly, in certain aspects, the present disclosure provides a therapeutic formulation comprising a dry powder (e.g., lyophilisate powder), wherein the dry powder comprises (i) one or more bacteria (e.g., those disclosed herein), (ii) urea, and (iii) one or more of the additional excipients disclosed herein.

In some embodiments, a therapeutic formulation disclosed herein is in a unit dosage form, each dosage form containing, e.g., from about 10² to about 10¹² colony forming units (CFUs) of bacteria per milligram (mg), for example, about 10⁴ to about 10¹⁰ CFUs of bacteria. In certain embodiments, a therapeutic formulation disclosed herein is in a unit dosage form, each dosage form containing, e.g., from 10² to 10¹² colony forming units (CFUs) of bacteria per milligram (mg), for example, 10⁴ to 10¹⁰ CFUs of bacteria. In other embodiments, a therapeutic formulation disclosed herein is in a multi-dose format. The therapeutic formulations of the present disclosure can be effective over a wide dosage range and is generally administered in a pharmaceutically effective amount.

In some embodiments, a therapeutic formulation of the present disclosure can be administered by a number of different means. In certain embodiments, a therapeutic formulation can be administered orally, rectally, parenterally, topically, or mucosally (e.g., oral mucosa), in formulations containing conventionally acceptable carriers, adjuvants, and vehicles as desired. The term “parenteral” as used herein includes subcutaneous, intravenous, intramuscular, or intrasternal injection and infusion techniques. In an exemplary embodiment, a therapeutic formulation is administered orally.

In some embodiments, a therapeutic formulation disclosed herein is administered to at least one region of the gastrointestinal tract, including the mouth, esophagus, stomach, small intestine, large intestine, rectum., and combinations thereof. In other embodiments, a formulation is administered to all regions of the gastrointestinal tract. In certain embodiments, a formulation is administered orally in the form of medicaments such as powders, capsules, tablets, gels, liquids, or combinations thereof. The formulation can also be administered in gel or liquid form by the oral route or through a nasogastric tube, or by the rectal route in a gel or liquid form, by enema or instillation through a colonoscope or by a suppository.

In some embodiments, a therapeutic formulation is provided in a dosage form. In some embodiments, the dosage form is designed for administration of at least one OTU or combination thereof disclosed herein, wherein the total amount of a therapeutic formulation administered is selected from about 0.1 ng to about 10 g, about 10 ng to about 1 g, about 100 ng to about 0.1 g, about 0.1 mg to about 500 mg, about 1 mg to about 1000 mg, from about 1000 mg to about 5000 mg, or more.

In some embodiments, a therapeutic formulation disclosed herein is administered to a subject (e.g., suffering from a disease or disorder disclosed herein) for at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, at least about 2 months, at least about 3 months, at least about 4 months, at least about 5 months, at least about 6 months, or at least about 1 year. In some embodiments, a therapeutic formulation is administered from about 1 day to about 1 week, from about 1 week to about 4 weeks, from about 1 month to about 3 months, from about 3 months to about 6 months, from about 6 months to about 1 year, or for over about a year.

In some embodiments, from about 10⁵ and about 10¹² microorganisms total is administered to the patient in a given dosage form of a therapeutic formation disclosed herein. In certain embodiments, an effective amount can be provided in from about 1 to about 500 ml or from about 1 to about 500 grams of the therapeutic formulation having from about 10⁷ to about 10¹¹ bacteria per ml or per gram, or a capsule, tablet, or suppository having from about 1 mg to about 1000 mg dry powder (e.g., lyophilisate powder) having from about 10⁷ to about 10¹¹ bacteria. In some embodiments, those receiving acute treatment receive higher doses than those who are receiving chronic administration (such as hospital workers or those admitted into long-term care facilities).

In some embodiments, a therapeutic formulation described herein is administered once, on a single occasion or on multiple occasions, such as once a day for several days or more than once a day on the day of administration (including twice daily, three times daily, or up to five times daily). In some embodiments, a therapeutic formulation is administered intermittently according to a set schedule. In other embodiments, a therapeutic formulation is administered on a long-term basis to individuals who are at risk a disease or disorder (e.g., those disclosed herein).

In some embodiments, a therapeutic formulation of the present disclosure is administered with other agents (e.g., anti-microbial agents or prebiotics) as a combination therapy mode. In certain embodiments, the administration is sequential, over a period of hours or days. In other embodiments, the administration is simultaneous. In other embodiments, the other agent (e.g. antibiotics) are administered as a pre-treatment.

In some embodiments, a therapeutic formulation is included in combination therapy with one or more anti-microbial agents, which include anti-bacterial agents, anti-fungal agents, anti-viral agents, and anti-parasitic agents, which can be administered separately as part of a dosing regimen.

Anti-bacterial agents include cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); carbapenem antibiotics (ertapenem, doripenem, imipenem/cilastatin, and meropenem); and combinations thereof. In some cases, an antibiotic agent is administered prior to treatment of a subject with a therapeutic formulation disclosed herein. In some embodiments, the anti-bacterial agent is administered to a subject and a therapeutic formulation is administered after the level of the anti-bacterial agent in the subject has reached a low enough level that it does not substantially affect viability of bacteria in the therapeutic formulation. In some embodiments, the anti-bacterial agent has little or no effect on viability of bacteria in the therapeutic formulation at the administered dose.

Examples of anti-viral agents include Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, Foscarnet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir, Zidovudine, and combinations thereof.

Examples of antifungal compounds include, but are not limited to, polyene antifungals such as natamycin, rimocidin, filipin, nystatin, amphotericin B, candicin, and hamycin; imidazole antifungals such as miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazole, terconazole, and albaconazole; thiazole antifungals such as abafungin; allylamine antifungals such as terbinafine, naftifine, and butenafine; echinocandin antifungals such as anidulafungin, caspofungin, and micafungin; and combinations thereof. Other compounds that have antifungal properties include, but are not limited to, polygodial, benzoic acid, ciclopirox, tolnaftate, undecylenic acid, flucytosine or 5-fluorocytosine, griseofulvin, haloprogin; and combinations thereof.

In some embodiments, a therapeutic formulation disclosed herein can be used to treat a subject with a microbiome-associated disease or disorders such as ulcerative colitis; Crohn's disease; lymphocytic colitis; microscopic colitis; collagenous colitis; autoimmune enteropathy; including autoimmune enteritis and autoimmune enterocolitis; allergic gastrointestinal disease; eosinophilic gastrointestinal disease, including eosinophilic gastroenteritis and eosinophilic enteropathy; and combinations thereof. In certain embodiments, the subject can have a dysbiosis, e.g., of the GI tract, an infection, be at risk for infection (for example, infection associated with antibiotic treatment, radiation, chemotherapy), or have another disease or disorder affected by the microbiome (for example, an inflammatory bowel disease, obesity, diabetes, asthma/allergy, an autoimmune disease, a central nervous system (CNS) disease or disorder (e.g., Autism Spectral Disorder (ASD) or Parkinson's Disease), a cholestatic disease, gastric ulcers, chronic heart diseases, rheumatic diseases, kidney diseases, or cancer).

### VI. Definitions

Certain terms used in the present application are defined as follows. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, 9 percent, 8 percent, 7 percent, 6 percent, 5 percent, 4 percent, 3 percent, 2 percent, or 1 percent; up or down (higher or lower).

The term “microbiota” refers to the ecological community of microorganisms that occur (sustainably or transiently) in and on an animal subject, typically a mammal such as a human, including eukaryotes, archaea, bacteria, and viruses (including bacterial viruses, i.e., phage).

The term “microbiome” refers to the microbes that live in and on the human body, both sustainably and transiently, including eukaryotes, archaea, bacteria, and viruses (including bacterial viruses (i.e., phage)). As used herein, “genetic content” includes genomic DNA, RNA such as ribosomal RNA, the epigenome, plasmids, and all other types of genetic information.

The term “dysbiosis” refers to a state of the microbiota of the GI tract or other body area in a subject, including mucosal or skin surfaces in which the normal diversity and/or function of the ecological network is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health.

As used herein, the term “operational taxonomic units” or “OTU” (or plural, “OTUs”) refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence can be the 16S rDNA sequence or a portion of the 16S rDNA sequence. In other embodiments, the entire genomes of two entities are sequenced and compared. In another embodiment, select regions such as multilocus sequence tags (MLST), specific genes, or sets of genes can be genetically compared. In 16S embodiments, OTUs that share 97% average nucleotide identity across the entire 16S or a variable region of the 16S rDNA, e.g., a V4 region, are considered the same OTU (see, e.g., Claesson, M. J., et al., Nucleic Acids Res 38: e200 (2010); Konstantinidis, K. T., et al., Philos Trans R Soc Lond B Biol Sci 361: 1929-1940 (2006)). In embodiments involving the complete genome, MLSTs, specific genes, or sets of genes OTUs that share 95% average nucleotide identity are considered the same OTU (see, e.g., Achtman, M. and Wagner M., Nat. Rev. Microbiol. 6: 431-440 (2008); Konstantinidis, K. T., et al., Philos Trans R Soc Lond B Biol Sci 361: 1929-1940 (2006)). OTUs are frequently defined by comparing sequences between organisms. Generally, sequences with less than 95% sequence identity are not considered to form part of the same OTU. In some cases, an OTU is characterized by a combination of nucleotide markers, genes, and/or single nucleotide variants (SNVs). In some cases, the referenced genes are highly conserved genes (e.g., “house-keeping” genes). The features defining an OTU can be a combination of the foregoing. Such characterization employs, e.g., WGS data or a whole genome sequence.

As used herein, the term “phylogenetic tree” refers to a graphical representation of the evolutionary relationships of one genetic sequence to another that is generated using a defined set of phylogenetic reconstruction algorithms (e.g., parsimony, maximum likelihood, or Bayesian). Nodes in the tree represent distinct ancestral sequences and the confidence of any node is provided by a bootstrap or Bayesian posterior probability, which measures branch uncertainty.

A “combination” of two or more bacteria includes the physical co-existence of the two bacteria, either in the same material or product or in physically connected products, as well as the temporal co-administration or co-localization of the two bacteria.

A “biologically pure culture” is a culture a culture of bacteria in a medium in which only selected viable species are present and no other viable species of microorganisms are detected.

The terms “lyophilisate,” “lyophilisate powder,” “lyophilized product,” “freeze-dried,” “dry powder,” and “product cake,” as used herein, denote a formulation/product which is manufactured by drying methods disclosed herein. In some embodiments, the terms can be used interchangeably.

As used herein, the term “lyophilizing” refers to the entire process of lyophilization, including both the freezing steps and the drying steps.

In lyophilization, water present in a material is converted to ice during a freezing step and then removed from the material by direct sublimation under low-pressure conditions during a primary drying step. During freezing, however, not all of the water is transformed to ice. Some portion of the water is trapped in a matrix of solids containing, for example, formulation components and/or the active ingredient. The excess bound water within the matrix can be reduced to a desired level of residual moisture during a secondary drying step. All lyophilization steps, freezing, primary drying and secondary drying, are determinative of the final product properties. The primary drying is typically the longest step in a lyophilization process, therefore, optimization of this portion of the process has significant economic effect.

As used herein, the term “spray drying” refers to a process involving breaking up liquid mixtures (e.g., bacterial compositions disclosed herein) into small droplets (atomization) and rapidly removing solvent from the mixture in a container (drying chamber) where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. This is accomplished by (1) mixing the liquid droplets with a warm drying gas, (2) maintaining the pressure in the spray-drying apparatus at a partial vacuum (e.g., 0.01 atm to 0.50 atm), or (3) both. See, e.g., U.S. Pat. No. 9,248,548, which is herein incorporated by reference in its entirety.

As used herein, the term “spray-freeze drying” refers to a process in which a liquid mixture (e.g., bacterial compositions disclosed herein) is atomized (i.e., broken up into small droplets) into a low temperature or cryogenic medium, such as liquid nitrogen, to obtain frozen droplets of the mixture, which can then be dried, e.g., via lyophilization. See, e.g., WO 2009/015286, which is herein incorporated by reference in its entirety.

As used herein, the term “collapse temperature” refers to the product temperature during freeze drying above which product cake begins to lose its structure. Above the collapse temperature, product could experience slow sporadic bubbling, swelling, foaming, cavitation, fenestration, gross collapse, retraction and beading that may have consequences on the appearance of the product. As a result, collapse may result in poor product stability, long drying times, uneven drying and loss of texture. See, e.g., U.S. Publ. No. 2010/0041870.

The term “accelerated stability,” as used herein, refers to the stability of a drug or product stored at elevated stress conditions (e.g., increased temperature). In many cases, it may not be feasible to test the long-term stability (e.g., >2 years) of a drug or product under real storage conditions. Therefore, by understanding the relationship between acceleration stability factor (e.g., increased temperature) and the degradation rate, it is possible to predict the degradation of the drug or product at the recommended storage conditions. In some embodiments, accelerated stability condition of 2 weeks at 30° C. can be used to predict the stability of the drug or product for approximately 1 year at 4° C. In certain embodiments, prior to the real-time stability testing, the relationship between temperature and product degradation can be calculated (e.g., using Arrhenius equation), which can then be used to predict the shelf-life of the product drug prior to the testing.

For nucleic acids, the term “substantial homology” indicates that two nucleic acids, or designated sequences thereof, when optimally aligned and compared, are identical, with appropriate nucleotide insertions or deletions, in at least 80% (e.g., at least 80%) of the nucleotides, at least 90% to 95% (e.g., at least 90% to 95%), or at least 98% to 99.5% (e.g., at least 98% to 99.5%) of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% (e.g., at least 80%) of the amino acids, at least about 90% to 95% (e.g., at least 90% to 95%), or at least about 98% to 99.5% (e.g., at least 98% to 99.5%) of the amino acids.

The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (I Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the) XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov. Other methods of determining identity that are known in the art can be used.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, dog, cow, chickens, amphibians, reptiles, etc.

As used herein, the terms “ug” and “uM” are used interchangeably with “μg” and “μM,” respectively.

Various aspects described herein are described in further detail throughout the specification.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and can vary depending upon the desired properties sought to be obtained. At the very least, and not as an attempt to limit the application of the doctrine of equivalents to the scope of the claims, each numerical parameter should be construed in light of the number of significant digits and ordinary rounding approaches.

The specification is most thoroughly understood in light of the teachings of the references cited within the specification. The embodiments within the specification provide an illustration of embodiments and should not be construed to limit the scope. The skilled artisan readily recognizes that many other embodiments are encompassed. All publications and patents cited in this disclosure are incorporated by reference in their entirety. To the extent the material incorporated by reference contradicts or is inconsistent with this specification, the specification will supersede any such material. The citation of any references herein is not an admission that such references are prior art.

The following examples are offered by way of illustration and not by way of limitation.

### EXAMPLES

### Example 1: Comparison of a Composition Containing Urea, No Urea, and a Commercially-Available Microbial Freeze Drying Composition on the Short-Term Stability and Viability of Gram-Negative Bacteria

To assess the effect of urea on the stability and yield of bacteria during and after lyophilization, samples of a Gram-negative bacteria species, Bacteroides faecis, were lyophilized using constructed formulations (i.e., non-commercial formulations; “CFs”) containing urea (0.5%) or no urea; or a commercially-available microbial freeze drying composition (OPS Diagnostics' Microbial Freeze Drying Buffer, cat. #MFDB 500-06). See Table 1. Unless indicated otherwise, the lyophilization compositions provided in Table 1 were neutralized to pH 7.0 with NaOH (i.e., composition #1 and 4-11). The bacteria were fermented in a medium suitable for growth and the fermentation kinetics were monitored (pH, optical density at 600 nm and cell viability by flow cytometry—live/dead stain (SYTO 9 dye and propidium iodide)). Once the bacteria reached early stationary phase, samples of the bacteria suspension were removed, and then buffer exchange was performed by washing the bacteria 3 times by centrifugation and exchanging the fermentation medium with the formulation solution (see Table 1). Samples of the bacteria in formulation solution (200 μL) were added to 2 mL glass vials and lyophilized. After lyophilization, vials were sealed under nitrogen and stored at the appropriate temperature. At each time point (i.e., post-lyophilization, after 1 week at 30° C., or after 2 weeks at 30° C.), 2-3 sample vials were reconstituted with PBS, plated, incubated, and CFU analyzed.

TABLE 1

| Composition | Sucrose | Other | Hy-Case ^{®} | rHA^{*} | Ascorbic | | HEPES | Histidine & |
|---|---|---|---|---|---|---|---|---|
| # | (%) | sugar (%) | SF (%) | (%) | acid (%) | Additives | (pH 7) | pH |
| 1 | 15 | 0 | 1 | 1 | 1 | 0.25% cysteine HCl | 10 mM | 0 mM/ pH |
| | | | | | | | | 7.0 |
| 2 | 15 | 0 | 1 | 1 | 1 | 0.25% cysteine HCl | 0 mM | 25 mM/ pH |
| | | | | | | | | 6.5 |
| 3 | 15 | 0 | 1 | 1 | 1 | 0.25% cysteine HCl | 0 mM | 25 mM/ pH |
| | | | | | | | | 6.0 |
| 4 | 15 | 0 | 1 | 1 | 1 | 0.25% cysteine HCl + | 10 mM | 0 mM/ pH |
| | | | | | | 25 mM NaCl | | 7.0 |
| 5 | 15 | 0 | 1 | 1 | 1 | 0.25% cysteine HCl + | 10 mM | 0 mM/ pH |
| | | | | | | 25 mM KCl | | 7.0 |
| 6 | 15 | 0 | 1 | 1 | 1 | 0.25% cysteine HCl + | 10 mM | 0 mM/ pH |
| | | | | | | 25 mM Na2SO4 | | 7.0 |
| 7 | 15 | 0 | 1 | 1 | 1 | 0.25% cysteine HCl + | 10 mM | 0 mM/ pH |
| | | | | | | 25 mM KI | | 7.0 |
| 8 | 15 | 0 | 1 | 1 | 1 | 0.25% cysteine HCl + | 10 mM | 0 mM/ pH |
| | | | | | | 25 mM arginine | | 7.0 |
| 9 | 15 | 0 | 1 | 1 | 1 | 0.25% cysteine HCl + | 10 mM | 0 mM/ pH |
| | | | | | | 0.5% urea | | 7.0 |
| 10 | 7.5 | 7.5% | 1 | 1 | 1 | 0.25% cysteine HCl | 10 mM | 0 mM/ pH |
| | | trehalose | | | | | | 7.0 |
| 11 | 15 | 0 | 1 | 1 | 1 | 0.25% cysteine + | 10 mM | 0 mM/ pH |
| | | | | | | 0.5% pectin | | 7.0 |

| | |
|---|---|
| 12 | OPS Freeze Drying Buffer |

^{*}HA = human albumin

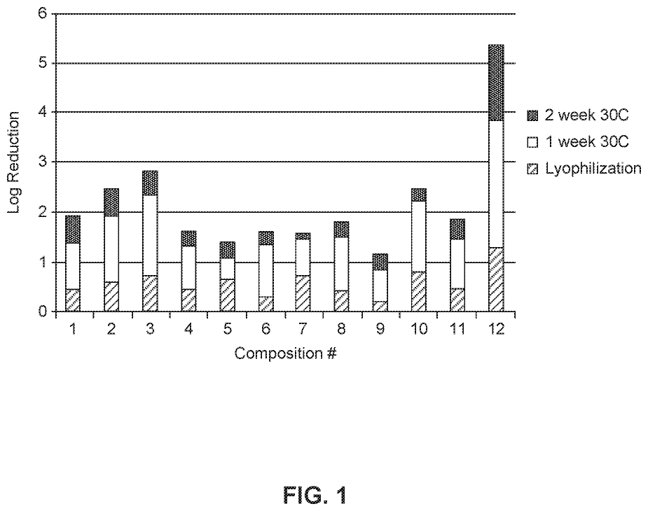

As illustrated in FIG. 1, a Gram-negative bacterium, Bacteroides faecis, lyophilized in the presence of 0.5% urea (composition 9) generally had greater lyophilization yield and stability compared to those bacteria lyophilized in the absence of urea (compositions 1-8, 10, and 11). Overall, CFs (with or without urea) resulted in much improved lyophilization yield and stability both after 1 week and 2 weeks at 30° C. when compared to the commercially available freeze-dried composition (OPS Diagnostics' Microbial Freeze Drying Buffer, composition 12).

These data demonstrate that a composition containing urea, particularly at 0.5% (w/w), can be useful in improving the stability of Gram-negative bacteria when dried (e.g., via lyophilization). They also demonstrate the superiority of the disclosed constructed formulations, even in the absence of urea, compared to a commercially available freeze-dried formulation.

### Example 2: Evaluation of the Addition of Dextran 70 and/or Nutra Hydrolyzed Gelatin on the Short-Term Stability and Viability of Gram-Negative Bacteria Strain SPC 10450 Formulated in 0.5% (w/w) Urea

Dextran 70 and Nutra hydrolyzed gelatin can be collapse temperature modifiers. Baheti, A., et al., J Excipient and Food Chem 1(1): 41-54 (2010). To assess whether increasing the collapse temperature (and thereby accelerating the lyophilization process) has an effect on lyophilization yield and/or stability of bacteria after lyophilization, a Gram-negative bacterium, Bacteroides faecis was lyophilized (see Example 1) in compositions containing 0.5% (w/w) urea, with or without Dextran 70 (Pharmacosmos) and/or hydrolyzed gelatin (Nutra® hydrolyzed gelatin, Nutra Food Ingredients). See Table 2. After lyophilization, vials were sealed under nitrogen and stored at a selected temperature. At each time point (i.e., post-lyophilization, after 1 week at 30° C., or after 2 weeks at 30° C., 2-3 sample vials were reconstituted with phosphate PBS, plated, incubated, and analyzed for CFU.

TABLE 2

| | | | | | | | | | | HEPES |
|---|---|---|---|---|---|---|---|---|---|---|
| | | | | | | | | | | buffer |
| Composition | Sucrose | Hy-Case | Hydrolyzed | Ascorbic | KCl | DTPA | CaCl2 | Urea | Dextran | pH 7.0 |
| # | (%) | SF (%) | gelatin (%) | acid (%) | (mM) | (uM) | (uM) | (%) | 70 (%) | (mM) |
| 1 (base) | 15 | 1 | 0 | 1 | 25 | 0 | 0 | 0 | 0 | 50 mM |
| 2 | | | | | | 100 | 100 | | | |
| 3 | | 0 | 1 | | | 0 | 0 | | | |
| 4 | | | 2 | | | | | | | |
| 5 | | | 4 | | | | | | | |
| 6 | | | 1 | | | | | 0.5 | | |
| 7 | | | 2 | | | | | | | |
| 8 | | | 4 | | | | | | | |
| 9 | | | 1 | | | | | | 2.5 | |
| 10 | | | 2 | | | | | | | |
| 11 | | | 4 | | | | | | | |
| 12 (10× titer, | | 1 | 0 | | | | | 0 | 0 | |
| base)) | | | | | | | | | | |

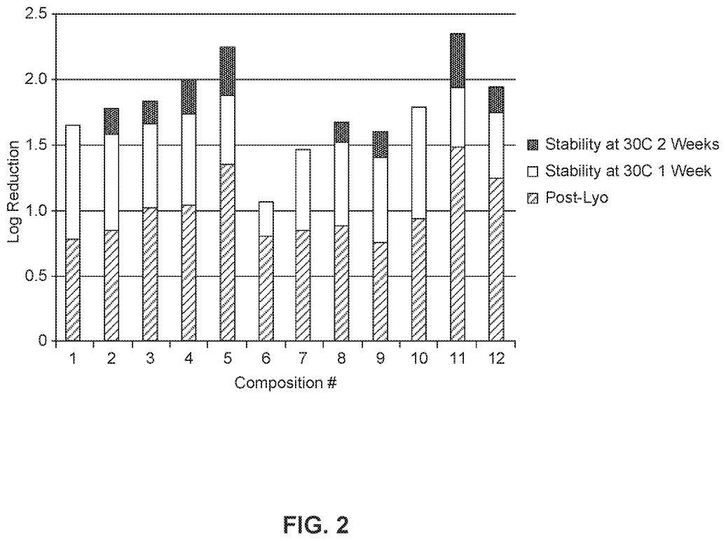

Similar to the results from Example 1, the addition of 0.5% urea to the lyophilization composition significantly improved both yield and stability. This was true across a range of gelatin concentrations (i.e., 1-4%) (see compositions 3, 4, 5 vs 6, 7, 8 in FIG. 2). The data further demonstrated that the addition of 2.5% (w/w) Dextran 70 to compositions that included 0.5% (w/w) urea and hydrolyzed gelatin had minimal effect on either lyophilization yield or stability. For example, compositions #9-11 (which contained Dextran 70) exhibited a log reduction of bacteria of about 1.5-2.5 after 2 weeks at 30° C. In contrast, compositions #6-8 (no Dextran 70) exhibited a log reduction of bacteria of about 1.0-1.5 after 2 weeks at 30° C. However, the addition of Dextran 70 did improve cake drying performance, with the best results observed with composition containing 0.5% (w/w) urea, 2.5% Dextran 70, and 2% hydrolyzed gelatin (composition #10).

Cake integrity is an important aspect of a drying process. Therefore, this result indicates that, although no significant effect was observed on bacteria stability, the use of Dextran 70 and hydrolyzed gelatin (e.g., Nutra®) can nonetheless be important when drying bacteria. Not to be bound by any one theory, collapse temperature modifiers may bind to different components found in the compositions disclosed herein (e.g., excipients), which can help remove the dried cake in one piece from the tray.

### Example 3: Comparison of AppliChem® Gelatin and Hydrolyzed Casein on the Short-Term Stability and Viability of Gram-Negative Bacteria Strain Bacteroides faecis Formulated in 0.5% (w/w) Urea

To further assess the effect of collapse temperature modifiers, such as gelatin, Gram-negative bacteria strain Bacteroides faecis was lyophilized (see Example 1) in a composition containing 0.5% (w/w) urea, with and without 1% AppliChem® gelatin (PanReac AppliChem). See Table 3. For comparison, compositions containing 0.5% (w/w) urea, with and without 1% hydrolyzed casein (Hy-Case SF) were also used.

TABLE 3

| | | | | | | | | HEPES |
|---|---|---|---|---|---|---|---|---|
| | | Ascorbic | Hy- | | | | | buffer |
| Composition | Sucrose | Acid | Case | Gelatin | KCl | Urea | Methionine | pH 7.0 |
| # | (%) | (%) | SF (%) | (%) | (mM) | (%) | (mM) | (mM) |
| 1 | 15 | 1 | 1 | 0 | 25 | 0.5 | 0 | 50 |
| 2 | | | 0 | 1 | | | | |
| 5 | | | 1 | 0 | | | 20 | |

| | |
|---|---|
| 6 (ADM + | Same as composition #1 (but the bacteria were grown in a different fermentation media) |
| Dextrose | |
| media) | |

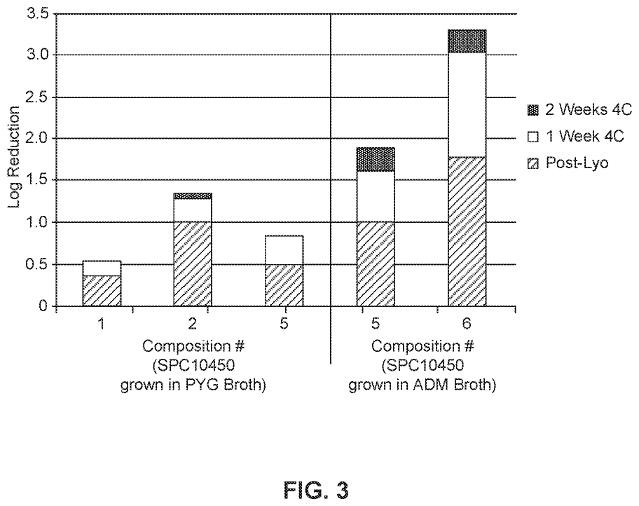

As shown in FIG. 3, the addition of 1% AppliChem gelatin did not improve bacteria stability (composition #2). Compositions that included 0.5% (w/w) urea but lacked AppliChem® gelatin (composition #1) had better bacteria stability (as evidenced by a mere half log loss over 2 weeks at 30° C.) compared to the composition containing gelatin, and also exhibited good cake properties (no cake collapse). Similarly, compositions that contained Hy-Case SF also resulted in much better stability and yield compared to compositions that contained AppliChem® gelatin.

These data indicate that the use of collapse temperature modifiers, for example AppliChem® gelatin, are not necessary and may be sub-optimal for generating stable bacteria with good cake properties after lyophilization.

### Example 4: Evaluation of a Composition Containing 0.5% (w/w) Urea on Viability Using Additional Bacterial Species

To assess whether a composition containing 0.5% (w/w) urea can also improve the viability of other types of bacteria (e.g., Gram-positive bacteria) during lyophilization, strains of Clostridium bolteae and Clostridium sp_D5 were lyophilized using the compositions shown in Table 5, and lyophilization yield was assessed.

TABLE 4

| | | | | | | | HEPES |
|---|---|---|---|---|---|---|---|
| Composition | Sucrose | Ascorbic | Hy-Case | VacciPro | | Urea | buffer pH 7.0 |
| # | (%) | Acid (%) | SF (%) | (%) | KCl (mM) | (%) | (mM) |
| 1 | 13 | 1 | 2 | 0 | 0 | 0 | 50 |
| 2 | | | | | | 0.5 | |
| 3 | | | | 3 | | | |
| 4 | 15 | | 1 | 0 | 25 | | |
| 5 | | | | 3 | | | |

| | |
|---|---|
| 6 | OPS Diagnostics Microbiological Freeze Drying Buffer (Cat. # MFDB 500-06) |

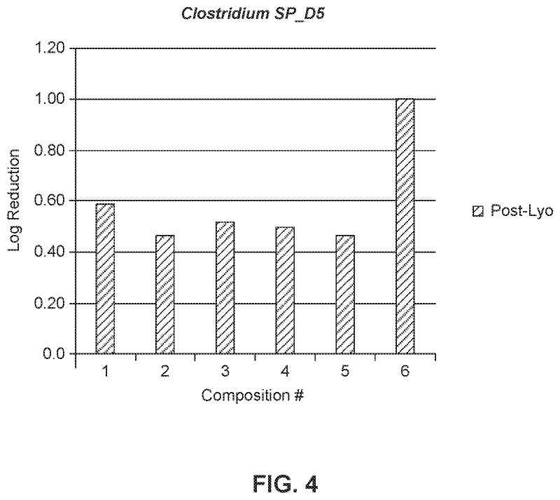

As shown in FIG. 4, the addition of 0.5% (w/w) urea improved the dry yield of Clostridium sp_D5. Without urea (composition #1), the log reduction was about 0.6. But, with urea (composition #2-5), the log reduction of Clostridium sp_D5 was reduced to about 0.5. Similarly, the lyophilization yield of Clostridium sp_D5 differed significantly when lyophilized with the commercially-available freeze-dried formulation (composition #6).

These data further confirm the benefit of using urea when drying bacterial compositions and demonstrate that the addition of urea can also improve the stability and dry yield of certain spore-forming bacteria.

### Example 5: Analysis of the Aerotolerance of Oxygen Sensitive Bacterial Strains Formulated in 0.5% Urea

To further assess the effect of a composition containing urea on bacteria during lyophilization, aerotolerance of two oxygen sensitive bacterial strains (Roseburia hominis and Eubacterium siraeum) was assessed after lyophilization.

Briefly, Roseburia Hominis and Eubacterium siraeum strains were fermented with a base media and carbon source optimized for growth. The fermentation was monitored using optical density at 600 nm, pH, and cell viability by flow cytometry with SYTO 9 dye and propidium iodide. When the bacteria reached early stationary phase, the fermentation was halted and the bacteria were buffer exchanged into a composition containing 10% or 12.5% sucrose, 1% ascorbic acid, 3% VacciPro, 25 mM KCl, 50 mM HEPES, and 0.5% urea at pH 7. The suspension of bacteria was then lyophilized. After lyophilization, the dried material was pouched for future analysis.

To determine the aerotolerance of the lyophilized bacteria, ˜100 mg aliquots of lyophilized material were apportioned into glass vials in an anaerobic environment. The vials were uncapped and removed from the anaerobic environment into the room air for the desired oxygen exposure time. Vials were then brought back into the anaerobic environment where the dry powder was reconstituted with pre-reduced PBS and titered with a standard colony forming unit assay. To determine the aerotolerance of non-lyophilized bacteria on an agar plate, bacteria were fermented in a media optimized for growth and then serially diluted in pre-reduced PBS. The diluted bacteria were spread on agar plates which were then exposed to oxygen for the determined amount of time. For each time point, the titer in CFU/mL of bacteria surviving was calculated.

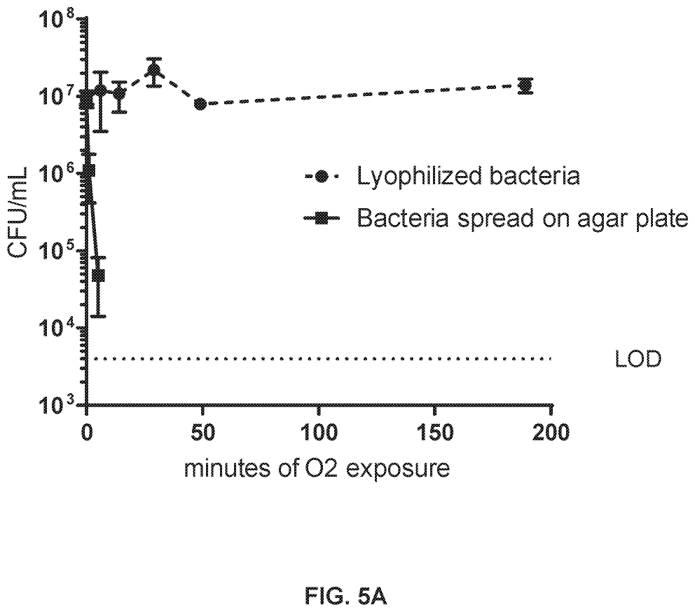

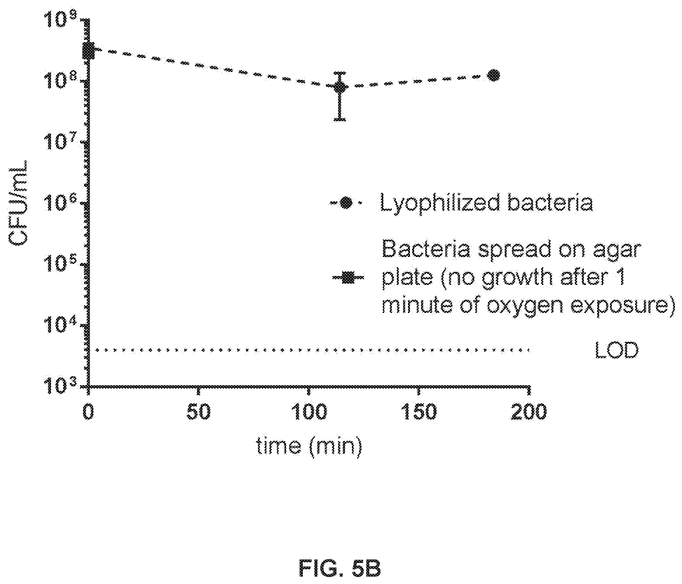

As shown in FIGS. 5A and 5B, the titer of both Eubacterium siraeum and Roseburia hominis strains, which were lyophilized in the above composition (i.e., containing 0.5% (w/w) urea), were maintained in the presence of oxygen for approximately three hours. In contrast, the non-lyophilized Eubacterium siraeum and Roseburia hominis strains were not able to survive upon oxygen exposure. This result highlights the additional benefit (i.e., increased aerotolerance) of using urea when drying bacterial compositions, particularly those that comprise oxygen-sensitive bacteria.

### Example 6: Demonstration of a Microbial Freeze Drying Composition Containing Urea to Stabilize Different Gram-Positive Bacteria

To demonstrate the universality of the freeze drying composition disclosed herein (e.g., comprising urea) on the stability and yield of bacteria after lyophilization, samples of bacterial strains representing the Gram-positive bacterial families Erysipelotrichaceae (two different strains), Ruminococcaceae (3 different strains), Lachnospiraceae and Eubacteriaceae were lyophilized using constructed formulations containing urea (0.5%). See Table 5. The compositions were neutralized to pH 7.0 with NaOH. The bacteria were fermented in a medium suitable for growth and the fermentation kinetics were monitored (pH and optical density at 600 nm). Once the bacteria reached early stationary phase, samples of the bacteria suspension were removed, and then buffer exchange was performed by washing the bacteria 2 times by centrifugation and exchanging the fermentation medium with the formulation solution (see Table 5). Samples of the bacteria in formulation solution (375 grams) were added to lyophilization trays and lyophilized. After lyophilization, the material was sampled, sealed under nitrogen and stored at the appropriate temperature. At each time point, a sample vial was reconstituted with Bovine Heart Infusion Solution (BHIS), plated, incubated, and CFU analyzed. The moisture content of the lyophilized bacteria formulations was also tested by Karl Fisher titration USP <921> in samples stored at ≤−65° C.

TABLE 5

| | Gelatin | Ascorbic | Potassium | HEPES | Urea | |
|---|---|---|---|---|---|---|
| Sucrose (%) | hydrolysate (%) | acid (%) | chloride (mM) | (mM) | (%) | NaOH |
| 10 | 3 | 1 | 25 | 50 | 0.5 | Adjust to pH |
| | | | | | | 7.0 |

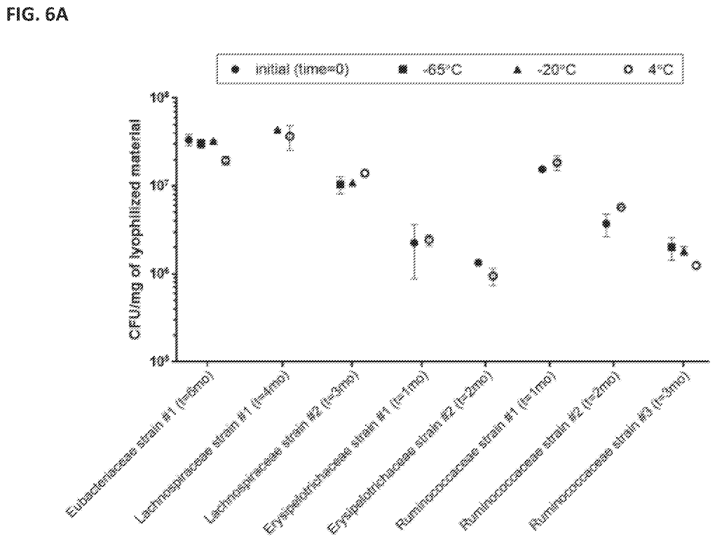

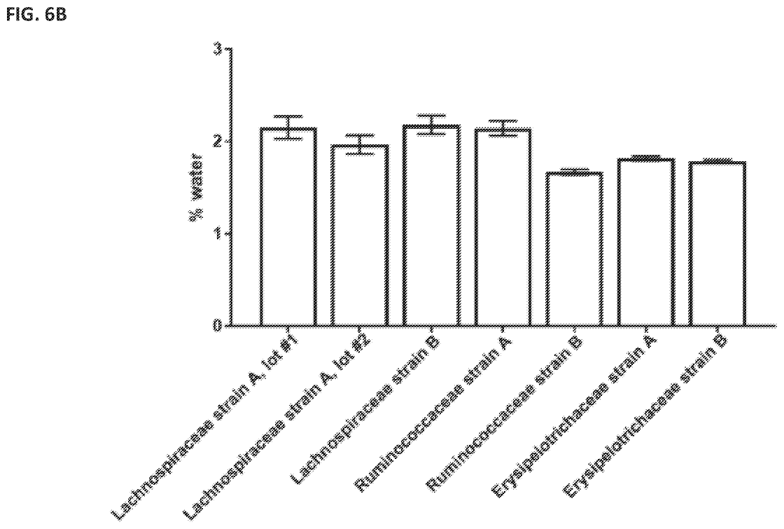

As illustrated in FIG. 6A, all the bacterial strains tested from the different Gram-positive bacterial families demonstrated stability at frozen (−65° C. and −20° C.), and refrigerated (4° C.) temperatures for up to 6 months. Moreover, as shown in FIG. 6B, bacteria compositions containing 0.5 urea could be lyophilized to form stabilized dry bacteria powder having moisture contents ranging from 1.7 to 2.2% water. Such a moisture level is typical of stable, lyophilized biologic drug products including peptides, proteins, antibodies, etc. Accordingly, these data demonstrate that a composition containing urea, particularly at 0.5% (w/w), can be useful to stabilize a wide range of Gram-positive bacteria when dried (e.g., via lyophilization).

What is claimed:

1. A composition comprising (i) one or more different OTUs of viable bacteria, (ii) urea, and (iii) one or more excipients selected from a cryoprotectant, an amino acid source, an antioxidant, a salt, a buffering agent, or combinations thereof.

2. The composition of claim 1, wherein the urea is present at a concentration (w/w) of between about 0.5% and about 1.0%.

3. The composition of claim 1 or 2, wherein the cryoprotectant is a sugar.

4. The composition of claim 3, wherein the sugar is a disaccharide.

5. The composition of claim 4, wherein the disaccharide is sucrose or trehalose.

6. The composition of claim 4, wherein the disaccharide is sucrose and trehalose.

7. The composition of claim 5 or 6, wherein the sucrose and/or trehalose is present at a concentration of between about 5% and about 20%.

8. The composition of any one of claims 1 to 7, wherein the amino acid source is a collagen.

9. The composition of claim 8, wherein the collagen is hydrolyzed collagen.

10. The composition of any one of claims 1 to 9, wherein the amino acid source is a gelatin.

11. The composition of claim 10, wherein the gelatin is a hydrolyzed gelatin.

12. The composition of claim 8 or 9, wherein the collagen is present at a concentration of about 3%.

13. The composition of claim 10 or 11, wherein the gelatin is present at a concentration between about 0.25% and about 4.0%.

14. The composition of any one of claims 1 to 13, wherein the amino acid source is a casein or an albumin.

15. The composition of claim 14, wherein the casein is hydrolyzed casein and/or the albumin is human serum albumin.

16. The composition of claim 14 or 15, wherein the casein and/or albumin is present at a concentration of about 1%.

17. The composition of any one of claims 1 to 16, wherein the antioxidant is cysteine.

18. The composition of any one of claims 1 to 16, wherein the antioxidant is ascorbic acid.

19. The composition of claim 17, wherein the cysteine is present at a concentration of about 0.25%.

20. The composition of claim 18, wherein the ascorbic acid is present at a concentration of about 1.0%.

21. The composition of any one of claims 1 to 20, wherein the salt is a potassium salt.

22. The composition of claim 21, wherein the potassium salt is potassium chloride (KCl).

23. The composition of claim 22, wherein the KCl is present at a concentration of about 25 mM.

24. The composition of any one of claims 1 to 23, wherein the buffering agent is 4-(2-hydroxyethyl)-1-piperazineethanesulfonic acid (HEPES).

25. The composition of claim 24, wherein the HEPES is present at a concentration between about 10 mM and about 100 mM.

26. The composition of any one of claims 1 to 25, wherein the viable bacteria are anaerobes.

27. The composition of claim 26, wherein the anaerobes have increased aerotolerance compared to corresponding anaerobes in a reference composition (e.g., lacks one of the excipients described herein, e.g., urea).

28. The composition of claim 26 or 27, wherein the anaerobes are facultative anaerobes.

29. The composition of claim 26 or 27, wherein the anaerobes are obligate anaerobes.

30. The composition of claim 26 or 27, wherein the anaerobes are aerotolerant anaerobes.

31. The composition of any one of claims 1 to 25, wherein the viable bacteria are aerobes.

32. The composition of any one of claims 1 to 31, comprising at least two OTUs of viable bacteria, wherein the at least two OTUs of viable bacteria comprises at least one facultative anaerobe, at least one obligate anaerobe, and/or at least one aerobe.

33. The composition of claim 32, comprising at least one anaerobe (e.g., aerotolerant anaerobes) and at least one aerobe.

34. The composition of any one of claims 1 to 33, wherein the viable bacteria are spore-forming bacteria.

35. The composition of any one of claims 1 to 34, wherein the viable bacteria are in a spore form.

36. The composition of any one of claims 1 to 34, wherein the viable bacteria are in a vegetative form.

37. The composition of any one of claims 1 to 34, wherein the viable bacteria are in a mixture of spore-form and vegetative-form.

38. The composition of any one of claims 1 to 37, wherein the viable bacteria are from one or more of the families Ruminococcaceae, Lachnospiraceae, Sutterellaceae, Clostridiaceae, Erysipelotrichaceae, Bacteroidaceae, Akkermansiaceae, Bifidobacteriaceae, Coriobacteriaceae, Enterobacteriaceae, Oscillospiraceae, Peptostreptococcaceae, Rikenellaceae, Streptococcaceae, or Desulfovibrionaceae.

39. The composition of any one of claims 1 to 38, wherein the viable bacteria comprise a 16S rDNA sequence that is at least 97% identical to the 16S rDNA sequence set forth in SEQ ID NOs: 1-368.

40. A dry powder comprising the composition of any one of claims 1 to 39.

41. The dry powder of claim 40, wherein the viable bacteria are stable for at least 1 week, at least 2 weeks, at least 3 weeks, at least 1 month, at least 2 months, at least 3 months, at least 6 months, at least 1 year, or at least 2 years.

42. The dry powder of claim 40 or 41, wherein the dry powder is encapsulated.

43. The dry powder of any one of claims 40 to 42, wherein the dry powder is reconstituted.

44. The dry powder of any one of claims 40 to 43, wherein the dry powder is used to treat a gastrointestinal disorder.

45. A therapeutic formulation comprising a dry powder of any one of claims 40 to 44.

46. The therapeutic formulation of claim 45, wherein the therapeutic formulation is administered orally, rectally, parenterally, topically, or mucosally.

47. The therapeutic formulation of claim 45 or 46, wherein the therapeutic formulation is used to treat a subject with a microbiome-associated disease or disorder.

48. The therapeutic formulation of claim 47, wherein the microbiome-associated disease or disorder comprises an inflammatory bowel disease, bacterial infection (e.g., Clostridium difficile infection), obesity, diabetes, asthma/allergy, an autoimmune disease, a central nervous system (CNS) disease or disorder (e.g., Autism Spectral Disorder (ASD) and Parkinson's Disease), a cholestatic disease, gastric ulcers, chronic heart diseases, rheumatic diseases, kidney diseases, cancer, or any combination thereof.
